(19) United States
(12) Patent Application Publication
(10) Pub. No.: US 2023/0108820 A1
(43) Pub. Date: Apr. 6, 2023

(54) FLUORINE-CONTAINING ETHER COMPOUND, FLUORINE-CONTAINING ETHER COMPOSITION, COATING LIQUID, HIGH OXYGEN SOLUBILITY LIQUID, AND ARTICLE

(71) Applicant: AGC Inc., Tokyo (JP)

(72) Inventor: Motoshi AOYAMA, Chiyoda-ku (JP)

(73) Assignee: AGC Inc., Tokyo (JP)

(21) Appl. No.: 18/052,996

(22) Filed: Nov. 7, 2022

Related U.S. Application Data
(63) Continuation of application No. PCT/JP2021/018254, filed on May 13, 2021.

(30) Foreign Application Priority Data
May 20, 2020 (JP) 2020-088258

(51) Int. Cl. C07C 43/12; C08L 71/08; C09D 171/08

(52) U.S. Cl. CPC C07C 43/12; C08L 71/08; C09D 171/08

(57) ABSTRACT

A fluorine-containing ether compound according to the present invention is expressed by the following formula (1) or the following formula (2):

Q¹{—(R^{f12})ₘ₂—O—(R^{f11}O)ₘ₁-A¹}ₙ₁ Formula (1)

{A¹-(OR^{f11})ₘ₁—O—(R^{f12})ₘ₂-}ₙ₂Q²-[(R^{f12})ₘ₂—O—(R^{f11}O)ₘ₁—(R^{f12})ₘ₂-Q³]ₚ—(R^{f12})ₘ₂—O—(R^{f11}O)ₘ₁—(R^{f12})ₘ₂-Q²-{(R^{f12})ₘ₂—O—(R^{f11}O)ₘ₁-A¹}ₙ₂ Formula (2),

wherein Q¹, Q², Q³, R^{f11}, R^{f12}, A¹, n1, n2, m1, m2, and p are as described in the specification.

### INCORPORATION BY REFERENCE

This application is based upon and claims the benefit of priority from Japanese Patent Application No. 2020-88258 filed on May 20, 2020, and PCT application No. PCT/JP2021/018254 filed on May 13, 2021, the disclosure of which is incorporated herein in its entirety by reference.

### BACKGROUND

The present invention relates to fluorine-containing ether compounds, fluorine-containing ether compositions, coating liquids, high oxygen solubility liquids, and articles.

Fluorine-containing ether polymers excel in various properties, such as a low refractive index, a low dielectric constant, water repellency and oil repellency, heat resistance, chemical resistance, chemical stability, lubricity, oxygen solubility, or transparency, and are used in a variety of fields, including electric and electronic materials, semiconductor materials, optical materials, materials for medical use, or surface treatment agents.

For example, a fluorine-containing ether compound having a polyfluoropolyether chain exhibits, for example, high lubricity, high heat resistance, or high water repellency and oil repellency and is thus used favorably as oil or grease suitable for use in lubricants, surface treatment agents, or the like.

Meanwhile, a fluorine-containing ether polymer has high oxygen solubility, and its application as a high oxygen solubility liquid is expected in materials for medical use, such as artificial blood.

International Patent Publication No. WO2019/131677 discloses a method of manufacturing a fluorine-containing polymer that includes cyclic ether and indicates that this fluorine-containing polymer has properties such as high heat resistance.

### SUMMARY

As stated above, fluorine-containing ether compounds can provide various physical properties mentioned above, and thus the demand for a fluorine-containing ether compound that can be used in various environments is on the rise. In recent years, as machine components are becoming smaller and lighter and of higher performance, for example, their operating environment temperature is increasingly on the rise, and this leads to the demand for a fluorine-containing ether compound that further excels in heat resistance.

The present invention is directed to providing a fluorine-containing ether compound that excels in heat resistance, a high oxygen solubility liquid, a fluorine-containing ether composition and a coating liquid, with which a surface layer that excels in heat resistance can be formed, and an article provided with a surface layer that excels in heat resistance.

The present invention provides a fluorine-containing ether compound, a fluorine-containing ether composition, a coating liquid, a high oxygen solubility liquid, and an article having any of the following constitutions [1] to [8].

[1] A fluorine-containing ether compound expressed by the following formula (1) or the following formula (2):

Q¹{—(R^{f12})ₘ₂—O—(R^{f11}O)ₘ₁-A¹}ₙ₁ Formula (1)

{A¹-(OR^{f11})ₘ₁—O—(R^{f12})ₘ₂-}ₙ₂Q²-[(R^{f12})ₘ₂—O—(R^{f11}O)ₘ₁—(R^{f12})ₘ₂-Q³]ₚ—(R^{f12})ₘ₂—O—(R^{f11}O)ₘ₁—(R^{f12})ₘ₂-Q²-{(R^{f12})ₘ₂—O—(R^{f11}O)ₘ₁-A¹}ₙ₂ Formula (2),

wherein

Q¹ is a carbocycle having a carbon number k1 that may have a fluorine atom, an alkyl group, or a fluoroalkyl group as a substituent, and k1 is an integer no smaller than 3,

Q² is a carbocycle having a carbon number k2 that may have a fluorine atom, an alkyl group, or a fluoroalkyl group as a substituent, k2 is an integer no smaller than 3, and the plurality of Q² may be identical to or different from each other,

Q³ is a carbocycle having a carbon number k3 that may have a fluorine atom, an alkyl group, or a fluoroalkyl group as a substituent, and k3 is an integer no smaller than 3,

R^{f11} and R^{f12} are each independently a fluoroalkylene group having a carbon number of from 1 to 6, and when there are a plurality of R^{f11} or R^{f12}, the R^{f11} or R^{f12} may each independently be identical to or different from each other,

A¹ is a fluoroalkyl group having a carbon number of from 1 to 20 or —R^{f21}—C(═O)—NR¹R², and when there are a plurality of A¹, the plurality of A¹ may be identical to or different from each other,

R^{f21} is an alkylene group or a fluoroalkylene group, having a carbon number of from 1 to 6,

R¹ and R² are each independently a hydrogen atom, a fluorine atom, an alkyl group that may have a double bond or a fluoroalkyl group that may have a double bond,

n1 is an integer of from 1 to k1,

n2 is an integer of from 0 to (k2-1), and the plurality of n2 may be identical to or different from each other,

m1 is an integer of from 1 to 500, and the plurality of m1 may be identical to or different from each other,

m2 is each independently 0 or 1, and the plurality of m2 may be identical to or different from each other, and

p is an integer of from 0 to 100.

[2] The fluorine-containing ether compound of [1], wherein at least one of the plurality of m2 is 0.

[3] The fluorine-containing ether compound of [1] or [2], wherein the carbocycle is an aliphatic carbocycle.

[4] The fluorine-containing ether compound according to any one of [1] to [3], wherein a weight-average molecular weight Mw is no lower than 1,500.

[5] A fluorine-containing ether composition that includes one or more kinds of the fluorine-containing ether compound of any one of [1] to [4], and another fluorine-containing ether compound.

[6] A coating liquid that includes the fluorine-containing ether compound of [1] to [4] or the fluorine-containing ether composition of [5], and a liquid medium.

[7] A high oxygen solubility liquid that includes the fluorine-containing ether compound of [1] to [4], and a liquid medium.

[8] An article that includes a substrate having a surface with a surface layer formed by the fluorine-containing ether compound of [1] to [4] or the fluorine-containing ether composition of [5].

The above and other objects, features and advantages of the present disclosure will become more fully understood from the detailed description given hereinbelow and the accompanying drawings which are given by way of illustration only, and thus are not to be considered as limiting the present disclosure.

### DESCRIPTION OF EMBODIMENTS

The present invention provides a fluorine-containing ether compound that excels in heat resistance, a high oxygen solubility liquid, a fluorine-containing ether composition and a coating liquid, with which a surface layer that excels in heat resistance can be formed, and an article provided with a surface layer that excels in heat resistance.

In the present specification, a compound expressed by Formula (1) is referred to as Compound 1, and a compound expressed by Formula (2) is referred to as Compound 2. This convention applies in a similar manner to compounds expressed by other formulas.

In the present specification, the terms listed below are to be construed as follows.

“Polyfluoropolyether” includes one with one ether, that is, includes “polyfluoroether,” unless specifically indicated otherwise.

A “surface layer” means a layer formed on a surface of a substrate.

The number of repeating units of a polyfluoropolyether chain of a fluorine-containing ether compound is a mean value calculated upon obtaining the number of oxyfluoroalkylene units through ¹H-NMR and ¹⁹F-NMR.

The number-average molecular weight (Mn), the weight-average molecular weight (Mw), and the polydispersity index (Mw/Mn) of a fluorine-containing ether compound are each a value obtained in polystyrene equivalent through gel permeation chromatography (GPC).

“From” and “to” indicating a numerical value range mean that each includes the numerical value following it as the lower limit value or the upper limit value.

### [Fluorine-Containing Ether Compound]

A fluorine-containing ether compound according to the present invention (also referred to below as the present compound) is a compound expressed by the following formula (1) or the following formula (2).

Q¹{—(R^{f12})ₘ₂—O—(R^{f11}O)ₘ₁-A¹}ₙ₁ Formula (1)

{A¹-(OR^{f11})ₘ₁—O—(R^{f12})ₘ₂-}ₙ₂Q²-[(R^{f12})ₘ₂—O—(R^{f11}O)ₘ₁—(R^{f12})ₘ₂-Q³]ₚ—(R^{f12})ₘ₂—O—(R^{f11}O)ₘ₁—(R^{f12})ₘ₂-Q²-{(R^{f12})ₘ₂—O—(R^{f11}O)ₘ₁-A¹}ₙ₂ Formula (2)

In the above,

Q¹ is a carbocycle having a carbon number k1 that may have a fluorine atom, an alkyl group, or a fluoroalkyl group as a substituent, and k1 is an integer no smaller than 3,

Q² is a carbocycle having a carbon number k2 that may have a fluorine atom, an alkyl group, or a fluoroalkyl group as a substituent, k2 is an integer no smaller than 3, and the plurality of Q² may be identical to or different from each other,

Q³ is a carbocycle having a carbon number k3 that may have a fluorine atom, an alkyl group, or a fluoroalkyl group as a substituent, and k3 is an integer no smaller than 3,

R^{f11} and R^{f12} are each independently a fluoroalkylene group having a carbon number of from 1 to 6, and when there are a plurality of R^{f11} or R^{f12}, the R^{f11} or R^{f12} may each independently be identical to or different from each other,

A¹ is a fluoroalkyl group having a carbon number of from 1 to 20 or —R^{f21}—C(═O)—NR¹R², and when there are a plurality of A¹, the plurality of A¹ may be identical to or different from each other,

R^{f21} is an alkylene group or a fluoroalkylene group, having a carbon number of from 1 to 6,

R¹ and R² are each independently a hydrogen atom, a fluorine atom, an alkyl group that may have a double bond or a fluoroalkyl group that may have a double bond,

n1 is an integer of from 1 to k1,

n2 is an integer of from 0 to (k2-1), and the plurality of n2 may be identical to or different from each other,

m1 is an integer of from 1 to 500, and the plurality of m1 may be identical to or different from each other,

m2 is each independently 0 or 1, and the plurality of m2 may be identical to or different from each other, and

p is an integer of from 0 to 100.

Compound 1 above includes one carbocycle (Q¹) and a polyfluoropolyether chain having a fluoroalkyl group or an amide group (A¹) and has a structure in which the carbocycle and the polyfluoropolyether chain are linked to each other.

Compound 2 above has a structure in which two or more carbocycles are linked to each other with a polyfluoropolyether chain intervening therebetween and in which the two or more carbocycles (Q²) are linked to one or more polyfluoropolyether chains each having a fluoroalkyl group or an amide group (A¹).

The present compound 1 and the present compound 2 both include a carbocycle having no heteroatom and a polyfluoropolyether chain having a fluoroalkyl group or an amide group (A¹).

The present compound, as it has a ring structure within a molecule, turns out to be a fluorine-containing ether compound that has a high boiling point and excels against friction or the like. Furthermore, since this ring structure is a carbocycle having no heteroatom, the present compound excels in chemical stability or heat resistance, as compared to a fluorine-containing ether compound that includes cyclic ether or the like. The present compound, although it depends on the molecular weight, is a liquid-state (oil-state) compound having a relatively high boiling point. The present compound, for example, has a weight-average molecular weight of no lower than 1,000, preferably no lower than 1,200, or more preferably no lower than 1,500, and excels in heat resistance.

The present compound is a compound having a polyfluoropolyether chain. It has been found that, while the present compound has various properties of a fluorine-containing ether polymer, such as a low refractive index, a low dielectric constant, water repellency and oil repellency, heat resistance, chemical resistance, chemical stability, oxygen solubility, or transparency, a surface layer formed by use of the present compound has somewhat low lubricity. Therefore, when the present compound is used as a surface treatment agent for a housing of a small-sized device, such as a smartphone, the present compound can prevent such an electronic device from slipping down while the electronic device can be provided with fingerprint removability.

Carbocycles in Q¹, Q², and Q³ can each independently be an aromatic carbocycle or an aliphatic carbocycle, for example.

An aromatic carbocycle may be a monocyclic ring, such as a benzene ring, or a fused ring, such as a naphthalene ring or an anthracene ring. From the standpoint of lubricity or transparency of the present compound, a benzene ring is preferable.

From the standpoint of ease of synthesis or chemical stability, an aliphatic carbocycle has a carbon number (k1) of preferably from 3 to 8, more preferably from 3 to 6, or even more preferably from 4 to 6. A carbon atom constituting an aliphatic carbocycle may have a double bond or a triple bond.

Examples of a structure of a carbocycle include the following.

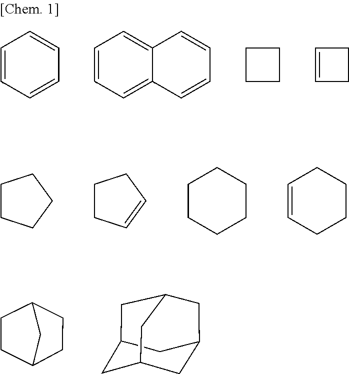

From the standpoint of heat resistance or the aforementioned various properties of the present compound as a fluorine-containing ether compound, carbocycles in Q¹, Q², and Q³ are preferably an aliphatic carbocycle of a carbon number (k1) of from 3 to 8, and in particular, a carbocycle having no double bond or triple bond is more preferable.

Carbocycles in Q¹, Q², and Q³ may have a fluorine atom, an alkyl group, or a fluoroalkyl group as a substituent.

An alkyl group is preferably a linear or branched alkyl group having a carbon number of from 1 to 5. Specific examples of an alkyl group include a methyl group, an ethyl group, or a tert-butyl group. A fluoroalkyl group can be one in which at least a part of a hydrogen atom in the alkyl group is substituted with fluorine.

From the standpoint of water repellency and oil repellency, a carbocycle preferably has a fluorine atom or a fluoroalkyl group as a substituent. Furthermore, from the standpoint of ease of synthesis or the like, a carbocycle preferably has a fluorine atom as a substituent. In other words, carbocycles in Q¹, Q², and Q³ are each preferably a perfluoro carbocycle or particularly preferably a perfluorocycloalkyl carbocycle.

The carbocycle Q¹ bonds with n1 polyfluoropolyether chains. Up to two polyfluoropolyether chains can bond with one carbon atom constituting the carbocycle Q¹. From the standpoint of heat resistance or ease of synthesis, in particular, it is preferable that one polyfluoropolyether chain bond with one carbon atom. From the standpoint of heat resistance or the like, n1 is preferably from 1 to 6 (herein, the upper limit is k1), or n1 is more preferably from 1 to 4 or even more preferably from 1 to 2.

The carbocycle Q² bonds with one polyfluoropolyether chain that is linked with at least another carbocycle and may further bond with a polyfluoropolyether chain having A¹. Up to two of these polyfluoropolyether chains can bond with one carbon atom constituting the carbocycle Q². From the standpoint of heat resistance or ease of synthesis, in particular, it is preferable that one polyfluoropolyether chain bond with one carbon atom. From the standpoint of heat resistance or the like, the number of bonds n2 of a polyfluoropolyether chain having A¹ is preferably from 0 to 5 (herein, the upper limit is (k2-1)), or n2 is more preferably from 0 to 3 or even more preferably from 0 to 1.

The carbocycle Q³ bonds with two polyfluoropolyether chains that are linked with another carbocycle. Up to two polyfluoropolyether chains can bond with one carbon atom constituting the carbocycle Q³. From the standpoint of heat resistance or the like, in particular, it is preferable that one polyfluoropolyether chain bond with one carbon atom.

In Formula (2), p represents the number of repetition of [(R^{f12})ₘ₂—O—(R^{f11}O)ₘ₁—(R^{f12})ₘ₂-Q³]. Compound 2 has (p+2) carbocycles. This p may be adjusted as appropriate in accordance with the intended use or the like of the present compound. From the standpoint of ease of synthesis, p is preferably from 0 to 80 or more preferably from 0 to 70.

Specific examples of Q¹, Q², and Q³ include those of the following formulas. Herein, * represents a bond with R^{f12} (when m2 is 1) or O (when m2 is 0). A fluorine atom in the following formulas may optionally be substituted by a hydrogen atom, an alkyl group, or a fluoroalkyl group.

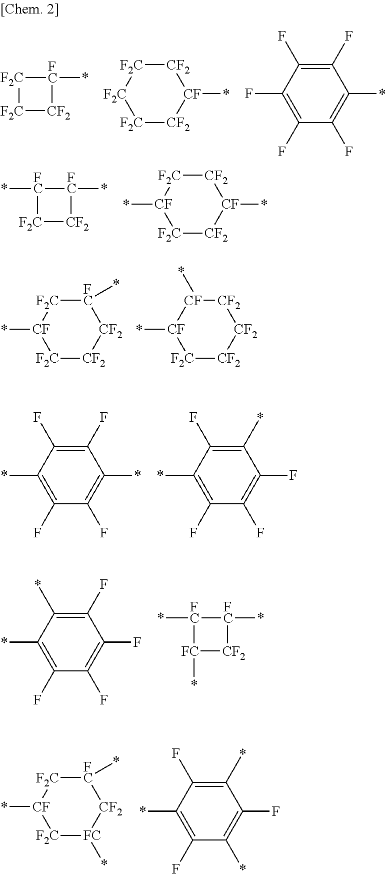

[(R^{f12})ₘ₂—O—(R^{f11}O)ₘ₁] represents a polyfluoropolyether chain.

R^{f12} is a fluoroalkylene group having a carbon number of from 1 to 6 and may be linear or branched. Specific examples of R^{f12} include —CF₂—, —CF₂CF₂—, —CF₂CF₂CF₂—, —CF₂CF₂CF₂CF₂—, —CF₂CF₂CF₂CF₂CF₂—, —CF₂CF₂CF₂CF₂CF₂CF₂—, or —CF₂CF(CF₃)—.

From the standpoint of suppressing steric hindrance with a ring structure and improving heat resistance, a fluoroalkylene group in R^{f12} is preferably a linear fluoroalkylene group having a carbon number of from 1 to 6, or more preferably a carbon number of from 1 to 4, or even more preferably a carbon number of from 1 to 2.

When m2 is 0, a carbocycle bonds with an oxygen atom in a polyfluoroether chain. Meanwhile, when m2 is 1, a carbocycle bonds with a carbon atom in a polyfluoroether chain. In either case, the present compound has various properties of a fluorine-containing ether compound. From the standpoint of heat resistance, m2 is preferably 0.

(R^{f11}O)ₘ₁ preferably has a structure expressed by the following formula (F1).

[(R¹¹O)ₘ₁₁(R¹²O)ₘ₁₂(R¹³O)ₘ₁₃(R¹⁴O)ₘ₁₄(R¹⁵O)ₘ₁₅(R¹⁶O)ₘ₁₆] Formula (F1)

In the above,

R¹¹ is a fluoroalkylene group having a carbon number of 1,

R¹² is a fluoroalkylene group having a carbon number of 2,

R¹³ is a fluoroalkylene group having a carbon number of 3,

R¹⁴ is a fluoroalkylene group having a carbon number of 4,

R¹⁵ is a fluoroalkylene group having a carbon number of 5,

R¹⁶ is a fluoroalkylene group having a carbon number of 6, and

m11, m12, m13, m14, m15, and m16 each represent 0 or an integer no smaller than 1, and (m11+m12+m13+m14+m15+m16) yields an integer of from 1 to 500.

Herein, the bonding order of (OR¹¹) to (OR¹⁶) in Formula (F1) is flexible. In Formula (F1), m11 to m16 represent the numbers of (OR¹¹) to (OR¹⁶), respectively, and do not represent their arrangement. For example, (OR¹⁵)ₘ₅ indicates that the number of (OR¹⁵) is m5 and does not represent the block arrangement structure of (OR¹⁵)ₘ₅. In a similar manner, the order in which (OR¹¹) to (OR¹⁶) are listed does not represent the bonding order of the respective units.

(R^{f11}O)ₘ₁ preferably has at least in part thereof a structure indicated below:

{(OCF₂)ₘ₂₁(OCF₂CF₂)ₘ₂₂},

(OCF₂CF₂)ₘ₂₃,

(OCF₂CF₂CF₂)ₘ₂₄,

(OCF₂CF₂—OCF₂CF₂CF₂CF₂)ₘ₂₅,

{(OCF₂CF₂CF₂)ₘ₂₆(OCF₂)ₘ₂₇},

{(OCF₂CF₂CF₂)ₘ₂₆(OCF₂CF₂)ₘ₂₇},

{(OCF₂CF₂CF₂CF₂CF₂)ₘ₂₆(OCF₂)ₘ₂₇},

{(OCF₂CF₂CF₂CF₂CF₂)ₘ₂₆(OCF₂CF₂)ₘ₂₇},

{(OCF₂CF₂CF₂CF₂CF₂CF₂)ₘ₂₆(OCF₂)ₘ₂₇},

{(OCF₂CF₂CF₂CF₂CF₂CF₂)ₘ₂₆(OCF₂CF₂)ₘ₂₇},

{(OCF₂CF(CF₃))ₘ₂₈(OCF₂)ₘ₂₉(OCF₂CF₂)ₘ₃₀},

{(OCF₂CF(CF₃))ₘ₂₈(OCF₂CF₂)ₘ₂₉(OCF₂)ₘ₃₀},

{(OCF₂CF(CF₃))ₘ₂₈(OCF₂CF₂CF₂)ₘ₂₉(OCF₂)ₘ₃₀},

{(OCF₂CF(CF₃))ₘ₂₈(OCF₂CF₂CF₂)ₘ₂₉(OCF₂CF₂)ₘ₃₀},

(OCF₂CF₂CF₂CF₂CF₂—OCF₂)ₘ₃₁,

(OCF₂CF₂CF₂CF₂CF₂—OCF₂CF₂)ₘ₃₁,

(OCF₂CF₂CF₂CF₂CF₂CF₂—OCF₂)ₘ₃₁,

(OCF₂CF₂CF₂CF₂CF₂CF₂—OCF₂CF₂)ₘ₃₁,

(OCF₂—OCF₂CF₂CF₂CF₂CF₂)ₘ₃₁,

(OCF₂—OCF₂CF₂CF₂CF₂CF₂CF₂)ₘ₃₁,

(OCF₂CF₂—OCF₂CF₂CF₂CF₂CF₂)ₘ₃₁,

(OCF₂CF₂—OCF₂CF₂CF₂CF₂CF₂CF₂)ₘ₃₁,

(OCF(CF₃)CF₂)ₘ₃₂, or

(OCF₂CF(CF₃))ₘ₃₂.

In the above, m21, m22, m23, m24, m25, m26, m27, m28, m29, m30, m31, and m32 are each an integer no smaller than 1, and the upper limit value is adjusted in accordance with the upper limit value of m1.

Herein, {(OCF₂)ₘ₂₁(OCF₂CF₂)ₘ₂₂} indicates that m21 (OCF₂) and m22 (OCF₂CF₂) are arranged at random.

From the standpoint of various properties of a fluorine-containing ether compound, such as chemical resistance or a low refractive index property, the polyfluoropolyether chain [(R^{f12})ₘ₂—O—(R^{f11}O)ₘ₁] preferably has a fluorination yield expressed by the following equation (1) of no lower than 60%, or more preferably no lower than 80%, or even more preferably substantially 100%, that is, being perfluoroether.

fluorination yield (%)=(number of fluorine atoms)/{(number of fluorine atoms)+(number of hydrogen atoms)}×100 Equation (1)

A¹ is a fluoroalkyl group having a carbon number of from 1 to 20 or —R^{f21}—C(═O)—NR¹R². Since A¹ has a fluoroalkyl group or a specific amide group, the present compound excels in heat resistance.

A fluoroalkyl group in A¹ may be linear or branched. A fluoroalkyl group may be selected as appropriate from those having a carbon number of from 1 to 20, in accordance with the intended use or the like. In particular, a carbon number of from 1 to 12 is preferable, a carbon number of from 1 to 6 is more preferable, or a carbon number of from 1 to 4 is even more preferable.

R^{f21} is an alkylene group or a fluoroalkylene group, having a carbon number of from 1 to 6 and may be linear or branched. Specific examples of an alkylene group having a carbon number of from 1 to 6 include —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, or —CH₂CH(CH₃)—. Examples of a fluoroalkylene group having a carbon number of from 1 to 6 include ones similar to those of the R^{f12} described above, and preferable embodiments are also similar.

R¹ and R² are each independently a hydrogen atom, a fluorine atom, an alkyl group that may have a double bond or a fluoroalkyl group that may have a double bond. The carbon number of the alkyl group and of the fluoroalkyl group is preferably from 1 to 6, more preferably from 1 to 4, or even more preferably from 1 to 3. Specific examples of an alkyl group that may have a double bond and specific examples of a fluoroalkyl group that may have a double bond include —CH₃, —CF₃, —CH₂CH₃, —CF₂CF₃, —CH═CH₂, —CF═CF₂, —CH₂CH₂CH₃, —CH₂CH═CH, or —CF₂CF═CF.

From the standpoint of excellent heat resistance, the weight-average molecular weight of the present compound is preferably no lower than 1,000, more preferably no lower than 1,200, or even more preferably no lower than 1,500. Meanwhile, although there is no particular limitation on the upper limit of the weight-average molecular weight, from the standpoint of ease of manufacture or the like, the weight-average molecular weight is preferably no higher than 500,000, more preferably no higher than 200,000, or even more preferably no higher than 100,000.

Meanwhile, in the present compound as a whole, the fluorination yield expressed by Equation (1) above is preferably no lower than 60%, more preferably no lower than 80%, or even more preferably no lower than 90%.

Specific examples of the present compound include the following compounds. Herein, o1 to o11 represent the number of repeating units and are adjusted as appropriate within a range of m1 (from 1 to 500).

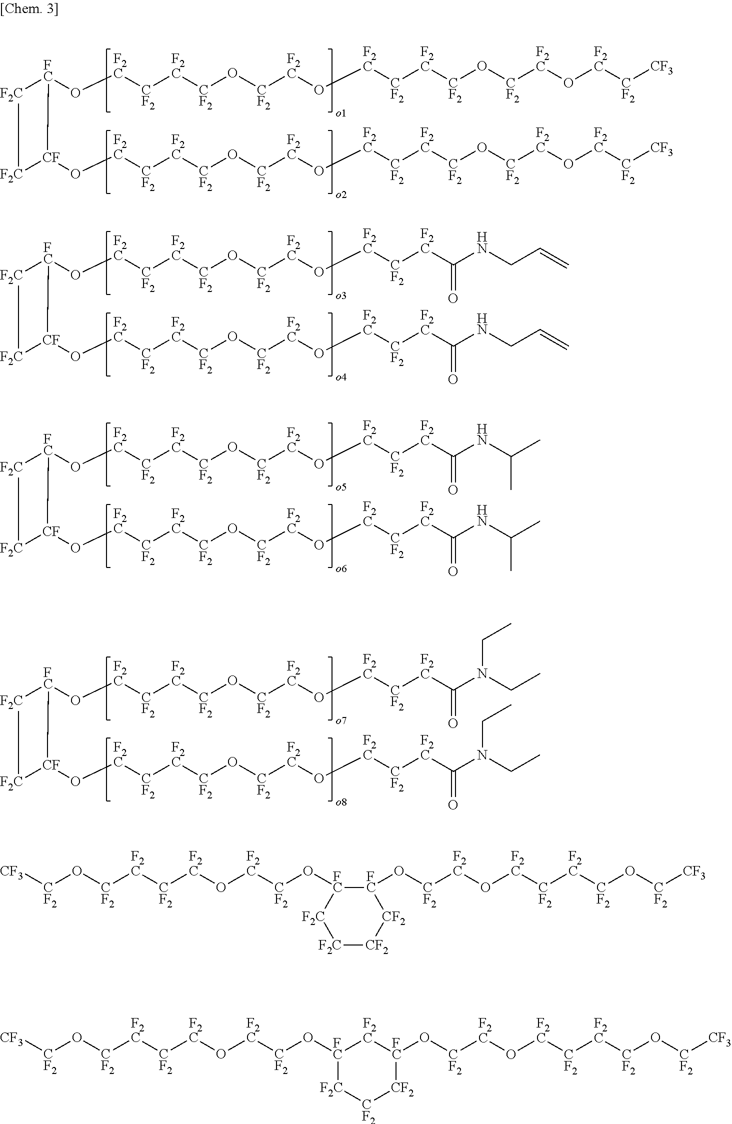

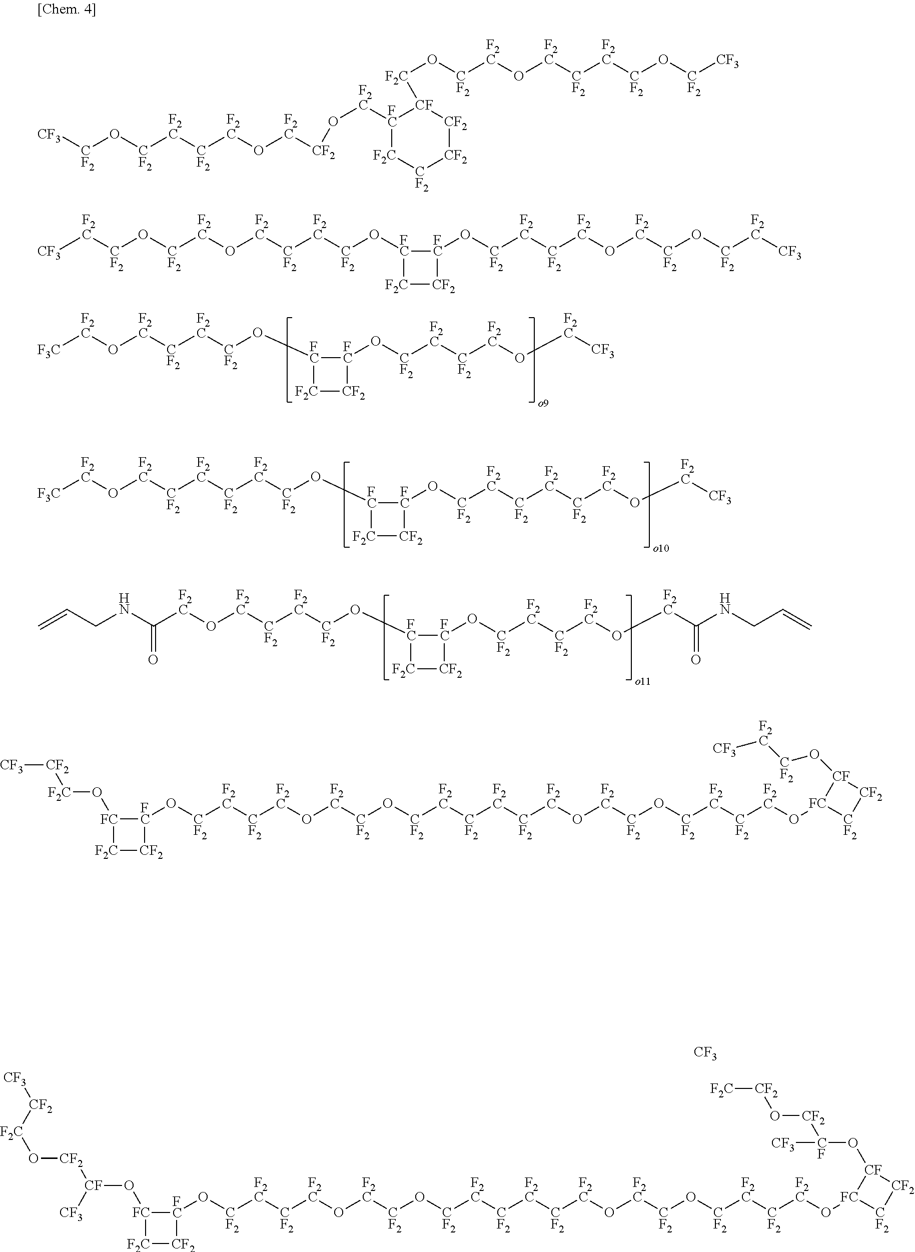

### <Method of Manufacturing the Present Compound>

Methods of manufacturing the present compound 1 include, for example, (I) a method in which a compound having a polyfluoroether chain is added to another compound having a carbocycle; or (II) a method in which two molecules of a compound having a polyfluoroether chain and one double bond are cycloadded.

Meanwhile, methods of manufacturing the present compound 2 include, for example, (III) a method in which two molecules of a compound having a carbocycle are linked to each other with a compound having a polyfluoroether chain intervening therebetween; or (IV) a method in which a compound having a polyfluoroether chain and two double bonds is subjected to cycloaddition polymerization. Each of these manufacturing methods will be described with examples below.

### (Method (I))

In one example of Method (I), Compound B1 below is reacted with Compound A¹ below to synthesize Compound 1.

Q¹(-(R^{f12})ₘ₂—OH)ₙ₁ Formula (A1)

CF₂═CF—(R^{f42})ₘ₄₂—O—(R^{f41}O)ₘ₄₁-A¹ Formula (B1)

Q¹{—(R^{f12})ₘ₂—O—(R^{f11}O)ₘ₁-A¹}ₙ₁ Formula (1)

In the above,

R^{f41} is a fluoroalkylene group having a carbon number of from 1 to 6, and when there are a plurality of R^{f41}, the R^{f41} may be identical to or different from each other,

R^{f42} is a fluoroalkylene group having a carbon number of from 1 to 4,

m41 is an integer of from 0 to 499,

m42 is 0 or 1, and

Q¹, R^{f11}, R^{f12}, A¹, m1, m2, and n1 are as described above, and their preferable embodiments are also as described above.

Herein, CF₂═CF—(R^{f42})ₘ₄₂O—(R^{f41}O)ₘ₄₁ of Compound B1 is a part that forms (R_{f11}O)ₘ₁ of Compound (1).

In Method (I), Compound 1 can be manufactured by charging a reaction vessel with Compound A1 and Compound B1 in the presence of a basic compound and by heating the resultant while adding pressure as necessary. The reaction temperature can be, for example, from 30° C. to 150° C. The reaction vessel and the reaction time may be adjusted as appropriate in accordance with the reaction scale or the like.

### (Method (II))

In one example of Method (II), two molecules of Compound B2 below are cycloadded to synthesize Compound 11.

CF₂═CF—(R^{f12})ₘ₂—O—(R¹¹O)ₘ₁-A¹ Formula (B2)

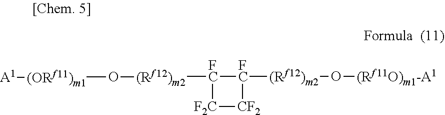

In the above,

R^{f11}, R^{f12}, A¹, m1, and m2 are as described above, and their preferable embodiments are also as described above.

Herein, two molecules of Compound B2 may be molecules identical to each other or two kinds of molecules between which any of R^{f11}, R^{f12}, A¹, m1, or m2 differs. When two molecules of Compound B2 are identical molecules, Compound 11 obtained is such that the plurality of R^{f11}, R^{f12}, A¹, m1, or m2 in Formula (11) are each identical to each other. When two molecules of Compound B2 are different from each other, Compound 11 obtained is such that at least one of the plurality of R^{f11}, R^{f12}, A¹, m1, or m2 in Formula (11) differs from each other.

In Method (II), Compound 11 can be manufactured, for example, by charging a reaction vessel with Compound B2 and heating Compound B2 while adding pressure as necessary. The reaction temperature can be, for example, from 30° C. to 250° C. The reaction vessel and the reaction time may be adjusted as appropriate in accordance with the reaction scale or the like.

### (Method (III))

In one example of Method (III), two molecules of Compound A2 below are reacted with Compound B3 below to synthesize Compound 21.

{A¹-(OR^{f11})ₘ₁—O—(R^{f12})ₘ₂-}ₙ₂Q²-(R^{f12})ₘ₂—OH Formula (A2)

CF₂═CF—(R^{f44})ₘ₄₄—O—(R^{f43}O)ₘ₄₃—(R^{f45})ₘ₄₅—CF═CF₂ Formula (B3)

{A¹-(OR^{f11})ₘ₁—O—(R^{f12})ₘ₂-}ₙ₂Q²-(R^{f12})ₘ₂—O—(R^{f11}O)ₘ₁—(R^{f12})ₘ₂-Q²-{(R^{f12})ₘ₂—O—(R^{f11}O)ₘ₁-A¹}ₙ₂ Formula (21)

In the above,

R^{f43} is a fluoroalkylene group having a carbon number of from 1 to 6, and when there are a plurality of R^{f43}, the R^{f43} may be identical to or different from each other,

R^{f44} and R^{f45} are each independently a fluoroalkylene group having a carbon number of from 1 to 4,

m43 is an integer of from 0 to 498,

m44 and m45 are each independently 0 or 1, and

Q², R^{f11}, R^{f12}, A¹, m1, m2, and n2 are as described above, and their preferable embodiments are also as described above.

Herein, Compound B3 is a part that forms (R^{f11}O)ₘ₁ of Compound 21 (the oxygen atom at the terminal is derived from Compound A2).

In Method (III), Compound 21 can be synthesized under a reaction condition similar to that of Method (I) described above.

### (Method (IV))

In one example of Method (IV), two or more molecules of Compound B4 below are subjected to thermal cycloaddition polymerization to synthesize Compound 22, and then fluorine is added to the resultant to synthesize Compound 23.

CF₂═CF—(R^{f12})ₘ₂—O—(R^{f11}O)ₘ₁—O—(R^{f12})ₘ₂—CF═CF₂ Formula (B4)

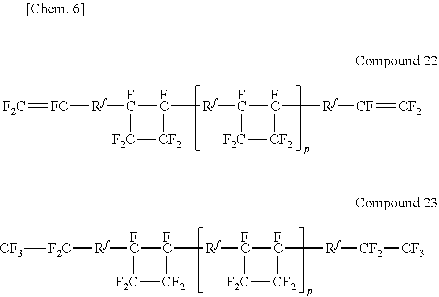

In the above,

R^{f} represents (R^{f12})ₘ₂—O—(R^{f11}O)ₘ₁—O—(R^{f12})ₘ₂; R^{f11}, R^{f12}, m1, m2, and p are as described above; and their preferable embodiments are also as described above.

In Method (IV), Compound 22 can be synthesized under a reaction condition similar to that of Method (II) described above.

Method (I) to Method (IV) described above are all examples. In a modification example, for example, a substituent that can be included in the present compound may be introduced into Compounds A1 and A2 and Compounds B1 to B4. Moreover, for example, a part of a polyfluoroether chain may be added to a compound having a carbocycle, and then the polyfluoroether chain may be stretched through polymerization of a known method.

### [Fluorine-Containing Ether Composition]

A fluorine-containing ether composition according to the present invention (also referred to below as the present composition) includes one or more kinds of the fluorine-containing ether compound which is the present compound, and another fluorine-containing ether compound other than the present compound. The present composition may include, as the present compound, both Compound 1 and Compound 2, for example. Herein, the present composition does not include a liquid medium described later.

Examples of another fluorine-containing ether compound include an inevitably included compound as well as a compound used in combination in accordance with the intended use or the like.

Examples of a compound used in combination with the present compound include, aside from known fluorine-containing oil, a fluorine-containing ether compound having a reactive silyl group or the like.

In a case where the present compound is used as a surface treatment agent, the present compound is preferably used in combination with a fluorine-containing ether compound having a reactive silyl group or the like. A fluorine-containing ether compound having a reactive silyl group or the like forms a surface layer that excels in adhesiveness as the reactive silyl group bonds with the surface of a substrate. Since the present compound excels in compatibility with a fluorine-containing ether compound having such a reactive silyl group or the like, the present compound is more easily retained in a surface layer, and the water repellency and oil repellency are more likely to be retained for an extended period of time.

Examples of fluorine-containing oil include polytetrafluoroethylene (PTFE), ethylene-chlorotrifluoroethylene copolymer (ECTFE), polyvinylidene fluoride (PVDF), polyvinyl fluoride (PVF), or polychlorotrifluoroethylene (PCTFE).

Meanwhile, examples of a fluorine-containing ether compound having a reactive silyl group or the like include a fluorine-containing ether compound commercially available as a surface treatment agent.

In a case where the present composition includes a known fluorine-containing ether compound, a novel effect of, for example, reinforcing the properties of the present compound may be exhibited.

Examples of a known fluorine-containing ether compound include those described in the following literatures:

Perfluoropolyether-modified aminosilane described in Japanese Unexamined Patent Application Publication No. H11-029585,

Silicon-containing organic fluoropolymer described in Japanese Patent No. 2874715,

Organic silicon compound described in Japanese Unexamined Patent Application Publication No. 2000-144097,

Perfluoropolyether-modified aminosilane described in Japanese Unexamined Patent Application Publication No. 2000-327772,

Fluorinated siloxane described in Published Japanese Translation of PCT International Publication for Patent Application, No. 2002-506887,

Organic silicone compound described in Published Japanese Translation of PCT International Publication for Patent Application, No. 2008-534696,

Fluoromodified hydrogenated polymer described in Japanese Patent No. 4138936,

Compound described in U.S. Patent Application Publication No. 2010/0129672, International Patent Publication No. WO2014/126064, or Japanese Unexamined Patent Application Publication No. 2014-070163,

Organosilicon compound described in International Patent Publication No. WO2011/060047 or International Patent Publication No. WO2011/059430,

Fluorine-containing organosilane compound described in International Patent Publication No. WO2012/064649,

Fluorooxyalkylene group-containing polymer described in Japanese Unexamined Patent Application Publication No. 2012-72272,

Fluorine-containing ether compound described in International Patent Publication No. WO2013/042732, International Patent Publication No. WO2013/121984, International Patent Publication No. WO2013/121985, International Patent Publication No. WO2013/121986, International Patent Publication No. WO2014/163004, Japanese Unexamined Patent Application Publication No. 2014-080473, International Patent Publication No. WO2015/087902, International Patent Publication No. WO2017/038830, International Patent Publication No. WO2017/038832, or International Patent Publication No. WO2017/187775,

Perfluoro(poly)ether-containing silane compound described in Japanese Unexamined Patent Application Publication No. 2014-218639, International Patent Publication No. WO2017/022437, International Patent Publication No. WO2018/079743, or International Patent Publication No. WO2018/143433,

Fluoropolyether group-containing polymer-modified silane described in Japanese Unexamined Patent Application Publication No. 2015-199906, Japanese Unexamined Patent Application Publication No. 2016-204656, Japanese Unexamined Patent Application Publication No. 2016-210854, or Japanese Unexamined Patent Application Publication No. 2016-222859, or

Fluorine-containing ether compound described in International Patent Publication No. WO2018/216630, International Patent Publication No. WO2019/039226, International Patent Publication No. WO2019/039341, International Patent Publication No. WO2019/039186, International Patent Publication No. WO2019/044479, Japanese Unexamined Patent Application Publication No. 2019-44158, or International Patent Publication No. WO2019/163282.

Meanwhile, examples of a commercially available fluorine-containing compound include the KY-100 series (KY-178, KY-185, KY-195, etc.) manufactured by Shin-Etsu Chemical Co., Ltd.; Afluid (registered trademark) S550 manufactured by AGC Inc.; or Optool (registered trademark) DSX, Optool (registered trademark) AES, Optool (registered trademark) UF503, or Optool (registered trademark) UD509 manufactured by Daikin Industries, Ltd.

In the present composition, in a case where a known fluorine-containing ether compound is combined with the present compound, the content ratio may be adjusted as appropriate in accordance with the intended use or the like. In particular, the content ratio of the present compound in the present composition is preferably from 10 mass % to 90 mass %, more preferably from 20 mass % to 80 mass %, or even more preferably from 25 mass % to 75 mass %. Setting the content ratio to the above range allows the properties of the present compound to be exhibited sufficiently and allows the properties of the fluorine-containing ether compound used in combination to be manifested sufficiently.

Examples of an inevitably included compound include a fluorine-containing ether compound produced as a by-product in the process of manufacturing the present compound (this by-product is also referred to below as a by-product fluorine-containing ether compound).

Examples of a by-product fluorine-containing ether compound include an unreacted fluorine-containing compound.

In a case where the present composition includes a by-product fluorine-containing ether compound, this by-product fluorine-containing ether compound can be removed through purification, but the by-product fluorine-containing ether compound may be contained in the present composition within a range in which the properties of the present compound can be exhibited sufficiently. Thus, the process of purifying the by-product fluorine-containing ether compound can be simplified.

In a case where no known fluorine-containing ether compound is combined, the content of the present compound in the present composition is, of the present composition, preferably no lower than 60 mass % but lower than 100 mass %, more preferably no lower than 70 mass % but lower than 100 mass %, or particularly preferably no lower than 80 mass % but lower than 100 mass %.

The content of a by-product fluorine-containing ether compound in the present composition is preferably higher than 0 mass % but no higher than 40 mass %, more preferably higher than 0 mass % but no higher than 30 mass %, or particularly preferably higher than 0 mass % but no higher than 20 mass %.

When the content of the present compound and the content of a by-product fluorine-containing ether compound are within the above ranges, further excellency can be observed in initial water repellency and oil repellency of a surface layer, friction resistance, fingerprint stain removability, slip resistance, light resistance, or chemical resistance.

### [Coating Liquid]

A coating liquid according to the present invention (also referred to below as the present coating liquid) includes the present compound or the present composition and a liquid medium. The present coating liquid may be a solution or a dispersion.

A liquid medium is preferably an organic solvent. An organic solvent may be a fluorine-containing organic solvent or a non-fluorinated organic solvent or may include both of such solvents.

Examples of a fluorine-containing organic solvent include fluorinated alkane, a fluorinated aromatic compound, fluoroalkylether, fluorinated alkylamine, or fluoroalcohol.

For fluorinated alkane, a compound having a carbon number of from 4 to 8 is preferable. Examples of commercially available products of such include C₆F₁₃H (manufactured by AGC Inc., Asahiklin (registered trademark) AC-2000), C₆F₁₃C₂H₅ (manufactured by AGC Inc., Asahiklin (registered trademark) AC-6000), or C₂F₅CHFCHFCF₃ (manufactured by The Chemours Company, Vertrel (registered trademark) XF).

Examples of a fluorinated aromatic compound include hexafluorobenzene, trifluoromethylbenzene, perfluorotoluene, or bis(trifluoromethyl)benzene.

For fluoroalkylether, a compound having a carbon number of from 4 to 12 is preferable. Examples of commercially available products of such include CF₃CH₂OCF₂CF₂H (manufactured by AGC Inc., Asahiklin (registered trademark) AE-3000), C₄F₉OCH₃(manufactured by 3M, Novec (registered trademark) 7100), C₄F₉OC₂H₅ (manufactured by 3M, Novec (registered trademark) 7200), or C₂F₅CF(OCH₃)C₃F₇ (manufactured by 3M, Novec (registered trademark) 7300).

Examples of fluorinated alkylamine include perfluorotripropylamine or perfluorotributylamine.

Examples of fluoroalcohol include 2,2,3,3-tetrafluoropropanol, 2,2,2-trifluoroethanol, or hexafluoroisopropanol.

For a non-fluorinated organic solvent, a compound consisting of hydrogen atoms and oxygen atoms or a compound consisting of hydrogen atoms, carbon atoms, and oxygen atoms is preferable, and examples of such include hydrocarbon, alcohol, ketone, ether, or ester.

A liquid medium may be a mixed medium in which two or more kinds are mixed together.

The content of the present compound or the present composition in the present coating solution is preferably from 0.001 mass % to 10 mass % or particularly preferably from 0.01 mass % to 1 mass %.

The content of a liquid medium in the present coating solution is preferably from 90 mass % to 99.999 mass % or particularly preferably from 99 mass % to 99.99 mass %.

### [High Oxygen Solubility Liquid]

A high oxygen solubility liquid according to the present invention (also referred to below as the present high oxygen solubility liquid) includes the present compound and a liquid medium.

Examples of a liquid medium include ones similar to those listed above for the coating liquid. Combining the present compound with a liquid medium provides a high oxygen solubility liquid that has high affinity with oxygen and excels in flowability.

The present high oxygen solubility liquid, as diluted with a large amount of water, is expected to be applied to artificial blood or the like.

### [Article]

An article according to the present invention (also referred to below as the present article) includes a substrate having a surface with a surface layer formed by the present compound or the present composition. A surface layer may be formed on a part of a surface of a substrate or on an entire surface of a substrate. A surface layer may spread like a membrane on a surface of a substrate or be present in dots.

A surface layer has a thickness of preferably from 1 nm to 100 nm or particularly preferably from 1 nm to 50 nm. When the thickness of a surface layer is no less than 1 nm, an advantageous effect from surface treatment is likely to be obtained sufficiently. When the thickness of a surface layer is no greater than 100 nm, the utilization efficiency is high. The thickness of a surface layer can be calculated by a vibration cycle of an interference pattern of reflected X-rays obtained through an X-ray reflectance technique by use of an X-ray diffractometer for thin films (manufactured by Rigaku Corporation, ATX-G).

Examples of a substrate include a substrate expected to be provided with heat resistance or slip resistance. Examples of such include a substrate that may be placed on another article (e.g., a holder), and as the surface layer described above is formed on a surface of that substrate where the substrate makes contact with another article, this article can be provided with excellent heat resistance and slip resistance.

Examples of a material for a substrate include metal, resin, glass, sapphire, ceramics, stone, or a composite material thereof. Glass may be chemically strengthened. A base film, such as a SiO₂ film, may be formed on a surface of a substrate.

Suitable examples of a substrate include a substrate for a touch panel, a substrate for a display, or an eyeglass lens, and a substrate for a touch panel is particularly suitable. Preferred materials for a substrate for a touch panel include glass or transparent resin.

The substrate is also preferably glass or a resin film used in exteriors of devices (excluding the display unit) such as mobile phones (such as smartphones), personal digital assistants (such as tablet terminals), game machines, and remote controllers.

### [Method of Manufacturing Article]

The present article can be manufactured, for example, through the following method.

In one method, a surface of a substrate is treated through a dry coating technique with use of the present compound or the present composition, and a surface layer formed by Compound 1 or Compound 2 or by the present composition is formed on the surface of the substrate (specifically, on the surface where the substrate makes contacts with another article).

In another method, a surface of a substrate is coated with the present coating solution through a wet coating technique and then dried, and a surface layer formed by the present compound or the present composition is formed on the surface of the substrate.

Examples of a dry coating technique include vacuum deposition, CVD, or sputtering. From the standpoint of suppressing decomposition of the present compound and the simplicity of a device, a vacuum deposition technique is preferable as a dry coating technique. In vacuum deposition, a pellet-form substance may be used in which a porous metal body of iron, steel, or the like is impregnated with the present compound or the present composition. A porous metal body of iron, steel, or the like may be impregnated with the present coating solution, the liquid medium may be dried out, and a pellet-form substance impregnated with the present compound or the present composition may be used.

Examples of a wet coating technique include a spin coating technique, a wipe coating technique, a spray coating technique, a squeegee coating technique, a dip coating technique, a die coating technique, an inkjet technique, a flow coating technique, a roll coating technique, a casting technique, a Langmuir-Blodgett technique, or a gravure coating technique.

### EXAMPLES

Hereinafter, the present invention will be described in further detail through examples, but the present invention is not limited by these examples. In the following, “%” means “mass %” unless specifically indicated otherwise. Examples 1 to 4, 7, 8, and 11 to 18 are examples pertaining to the present compound, and Examples 5, 6, 9, and 10 are comparative examples.

### Example 1

### Example 1-1

One hundred g of CF₂═CF—O—CF₂CF₂CF₂CF₂—O—CF═CF₂ (Compound 1-1) was loaded into a 250-mL metal reactor and stirred for 300 hours at 160° C. The temperature inside the reactor was brought to 25° C., and the obtained reaction liquid was subjected to thin film distillation at 130° C. under reduced pressure. Thus, a low-boiling component was removed, and 63 g of Compound 1-2 was obtained.

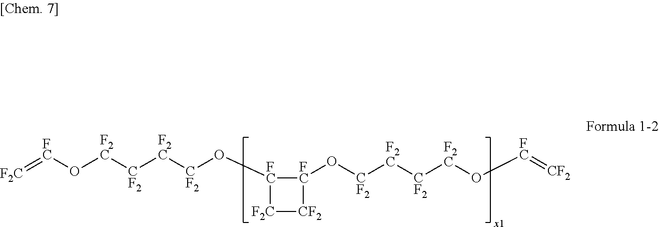

Mean value of x1: 10, Polydispersity index (Mw/Mn): 1.50

The NMR spectrum of Compound 1-2;

¹⁹F-NMR (282.7 MHz, solvent: CDCl₃, reference: CFCl₃) δ (ppm): −83 (44F), −116 (2F), −124 (46F), −128 (20F), −131 (20F), −137 (12F), −139 (10F).

### Example 1-2

Two hundred fifty mL of ClCF₂CFClCF₂OCF₂CF₂Cl (referred to below as CFE-419) was loaded into a 500-mL metal reactor, and after nitrogen gas was bubbled, fluorine gas of 20 volume % diluted with nitrogen gas was bubbled. A CFE-419 solution of Compound 1-2 obtained in Example 1-2 (concentration: 10%, Compound 1-2: 60 g) was introduced thereinto. The ratio of the introduction rate (mol/hour) of fluorine gas and the introduction rate (mol/hour) of hydrogen atoms in Compound 1-2 was controlled to 2:1. After Compound 1-2 was introduced, a CFE-419 solution of benzene (concentration: 0.1%, benzene: 0.1 g) was introduced intermittently. After benzene was introduced, fluorine gas was bubbled, and lastly the inside of the reactor was sufficiently replaced with nitrogen gas. The solvent was distilled, and 59 g of Compound 1-3 was obtained.

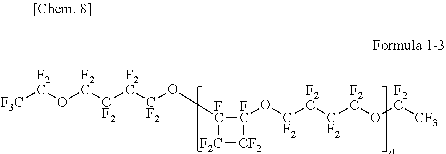

Mean value of x1: 10, Polydispersity index: 1.52

The NMR spectrum of Compound 1-3;

¹⁹F-NMR (282.7 MHz, solvent: CDCl₃, reference: CFCl₃) δ (ppm): −83 (48F), −87 (6F), −124 (44F), −128 (20F), −131 (20F), −137 (10F), −139 (10F).

### Example 2

### Example 2-1

One hundred g of CF₂═CF—O—CF₂CF₂CF₂CF₂CF₂CF₂—O—CF═CF₂ was loaded into a 250-mL metal reactor and stirred for 250 hours at 160° C. The temperature inside the reactor was brought to 25° C., and the obtained reaction liquid was subjected to thin film distillation at 130° C. under reduced pressure. Thus, a low-boiling component was removed, and 57 g of Compound 2-1 was obtained.

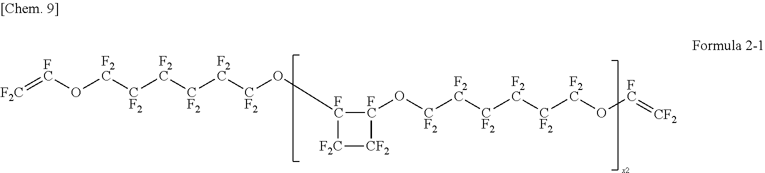

Mean value of x2: 8. Polydispersity index: 1,47

The NMR spectrum of Compound 2-1;

¹⁹F-NMR (282.7 MHz, solvent: CDCl₃, reference: CFCl₃) δ (ppm): −83 (36F), −116 (2F), −124 (36F), −127 (36F), −128 (16F), −131 (16F), −137 (10F), −139 (8F).

### Example 2-2

Fifty-nine g of Compound 2-2 was obtained in a manner similar to that in Example 1-3, except that Compound 1-2 was changed to 55 g of Compound 2-1 obtained in Example 2-1.

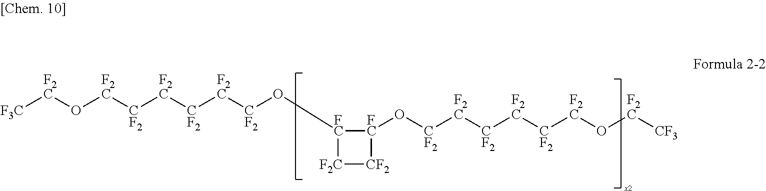

Mean value of x2: 8, Polydispersity index: 1.48

The NMR spectrum of Compound 2-2;

¹⁹F-NMR (282.7 MHz, solvent: CDCl₃, reference: CFCl₃) δ (ppm): −83 (40F), −87 (6F), −124 (34F), −127 (36F), −128 (16F), −131 (16F), −137 (8F), −139 (8F).

### Example 3

### Example 3-1

Compound 3-1 was obtained in accordance with the method described in Example 1-1 of Examples in International Patent Publication No. WO2013/121984.

CF₂═CF—O—CF₂CF₂CF₂CH₂—OH Formula 3-1

### Example 3-2

Thirty g of Compound 3-1 obtained in Example 3-1 was loaded into a 100-mL metal reactor and stirred at 175° C. The obtained organic phase was concentrated, and 18 g of Compound 3-2 was obtained.

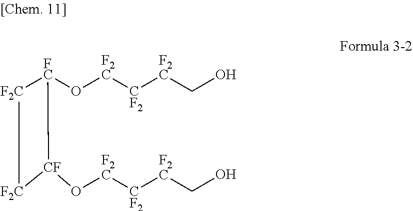

### Examples 3-3

Fifteen g of Compound 3-2 obtained in Example 3-2 and 3.6 g of potassium carbonate were loaded into a 200-mL recovery flask and stirred at 120° C. Then, 75 g of Compound 3-1 was added and stirred for 2 hours at 120° C. The temperature inside the recovery flask was brought to 25° C., and AC-2000 and hydrochloric acid were added and separated to concentrate an organic phase. The obtained reaction crude liquid was purified through column chromatography, and 63 g of Compound 3-3 was obtained.

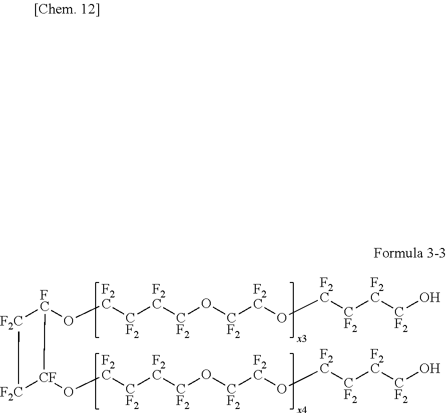

Mean value of x3+x4: 8

### Example 3-4

Fifty g of Compound 3-3, 14.3 g of CF₂═CFOCF₂CF₂CF₃, and 2.5 g of potassium carbonate were loaded into a 300-mL recovery flask and stirred for 2 hours at 40° C. Hydrochloric acid was added, and an organic phase obtained through separation was dehydrated with magnesium sulfate. Magnesium sulfate was filtered out, the crude liquid was concentrated, and 58 g of Compound 3-4 was obtained.

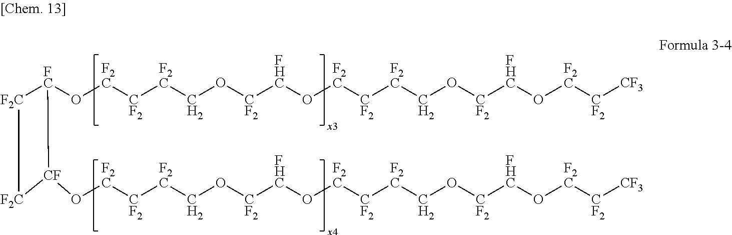

Mean value of x3+x4: 8, Polydispersity index: 1.40

The NMR spectrum of Compound 3-4;

¹H-NMR (300.4 MHz, solvent: CDCl₃, reference: TMS) δ (ppm): 6.0 (101H), 4.6 (20H).

¹⁹F-NMR (282.7 MHz, solvent: CDCl₃, reference: CFCl₃) δ (ppm): −81 (6F), −85 (24F), −91 (20F), −120 (16F), −126 (16F), −128 (2F), −129 (4F), −131 (2F), −137 (1F), −139 (1F), −144 (8F).

### Example 3-5

Two hundred fifty mL of CFE-419 was loaded into a 500-mL metal reactor, and after nitrogen gas was bubbled, fluorine gas of 20 volume % diluted with nitrogen gas was bubbled. A CFE-419 solution of Compound 3-4 (concentration: 10%, Compound 3-4: 50 g) was introduced thereinto. The ratio of the introduction rate (mol/hour) of fluorine gas and the introduction rate (mol/hour) of hydrogen atoms in Compound 3-4 was controlled to 2:1. After Compound 3-4 was introduced, a CFE-419 solution of benzene (concentration: 0.1%, benzene: 0.5 g) was introduced intermittently. After benzene was introduced, fluorine gas was bubbled, and lastly the inside of the reactor was sufficiently replaced with nitrogen gas. The solvent was distilled, and 56 g of Compound 3-5 was obtained.

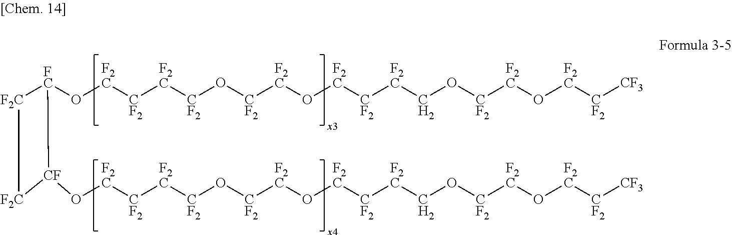

Mean value of x3+x4: 8, Polydispersity index: 1.38

The NMR spectrum of Compound 3-5;

¹⁹F-NMR (282.7 MHz, solvent: CDCl₃, reference: CFCl₃) δ (ppm): −81 (6F), −83 (44F), −87 (40F), −124 (40F), −128 (2F), −129 (4F), −131 (2F), −137 (1F), −139 (1F).

### Example 4

### Example 4-1

Fifty g of Compound 3-2 above, 71.7 g of CF₂═CFOCF₂CF₂CF₃, and 12.4 g of potassium carbonate were loaded into a 300-mL recovery flask and stirred for 2 hours at 40° C. Hydrochloric acid was added, and an organic phase obtained through separation was dehydrated with magnesium sulfate. Magnesium sulfate was filtered out, the crude liquid was concentrated, and 95.8 g of Compound 4-1 was obtained.

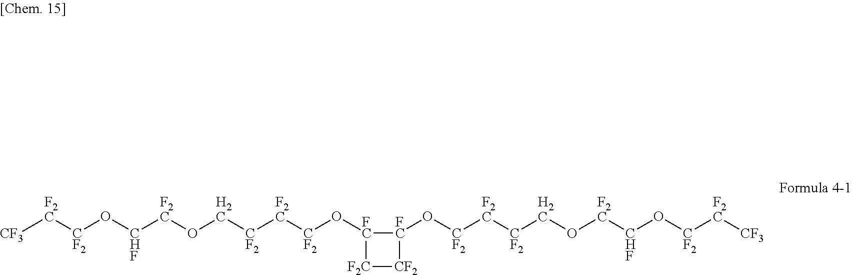

The NMR spectrum of Compound 4-1;

¹H-NMR (300.4 MHz, solvent: CDCl₃, reference: TMS) δ (ppm): 6.0 (2H), 4.6 (4H).

¹⁹F-NMR (282.7 MHz, solvent: CDCl₃, reference: CFCl₃) δ (ppm): −81 (6F), −85 (8F), −91 (4F), −120 (4F), −126 (4F), −128 (2F), −129 (4F), −131 (2F), −137 (1F), −139 (1F), −144 (2F).

### Example 4-2

Two hundred fifty mL of CFE-419 was loaded into a 500-mL metal reactor, and after nitrogen gas was bubbled, fluorine gas of 20 volume % diluted with nitrogen gas was bubbled. A CFE-419 solution of Compound 4-1 (concentration: 30%, Compound 4-1: 90 g) was introduced thereinto. The ratio of the introduction rate (mol/hour) of fluorine gas and the introduction rate (mol/hour) of hydrogen atoms in Compound 4-1 was controlled to 2:1. After Compound 4-1 was introduced, a CFE-419 solution of benzene (concentration: 0.1%, benzene: 0.1 g) was introduced intermittently. After benzene was introduced, fluorine gas was bubbled, and lastly the inside of the reactor was sufficiently replaced with nitrogen gas. The solvent was distilled, and 96 g of Compound 4-2 was obtained.

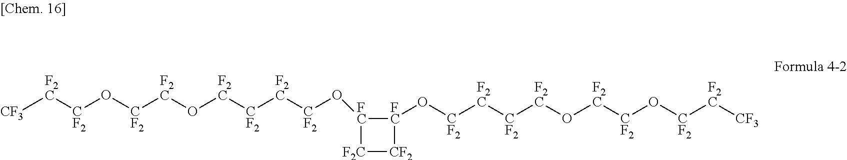

The NMR spectrum of Compound 4-2;

¹⁹F-NMR (282.7 MHz, solvent: CDCl₃, reference: CFCl₃) δ (ppm): −81 (6F), −83 (8F), −87 (4F), −124 (4F), −128 (2F), −129 (4F), −131 (2F), −137 (1F), −139 (1F), −144 (2F).

### Example 5

Compound 5-1 below (product name: Fomblin M03, manufactured by Solvay Solexis Inc., polydispersity index: 1.40) was prepared.

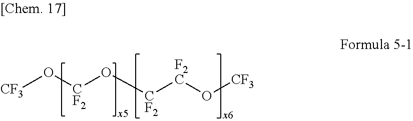

X6/x5=1.06, Numher-average molecular weight: 4028, Polydispersity index: 1.40

### Example 6

### Example 6-1

Compound 6-1 was obtained in accordance with a method described in International Patent Publication No. WO2017/038830.

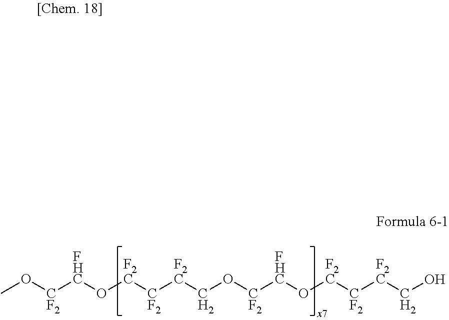

Mean value of x7: 9

### Example 6-2

Fifty g of Compound 6-1, 7.1 g of CF₂═CFOCF₂CF₂CF₃, and 2.5 g of potassium carbonate were loaded into a 300-mL recovery flask and stirred at 40° C. Hydrochloric acid was added, and an organic phase obtained through separation was dehydrated with magnesium sulfate. Magnesium sulfate was filtered out, the crude liquid was concentrated, and 58 g of Compound 6-2 was obtained.

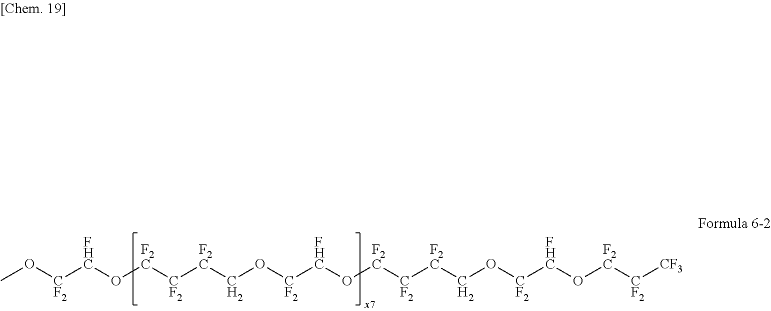

Mean value of x7: 9. Polydispersity index through GPC: 1.35

The NMR spectrum of Compound 6-2;

¹H-NMR (300.4 MHz, solvent: CDCl₃, reference: TMS) δ (ppm): 6.0 (11H), 4.6 (20H), 3.4 (3H).

¹⁹F-NMR (282.7 MHz, solvent: CDCl₃, reference: CFCl₃) δ (ppm): −81 (3F), −85 (22F), −91 (22F), −120 (20F), −126 (20F), −129 (2F), −144 (11F).

### Example 6-3

Two hundred fifty mL of CFE-419 was loaded into a 500-mL metal reactor, and after nitrogen gas was bubbled, fluorine gas of 20 volume % diluted with nitrogen gas was bubbled. A CFE-419 solution of Compound 6-2 (concentration: 10%, Compound 6-2: 50 g) was introduced thereinto. The ratio of the introduction rate (mol/hour) of fluorine gas and the introduction rate (mol/hour) of hydrogen atoms in Compound 6-2 was controlled to 2:1. After Compound 6-2 was introduced, a CFE-419 solution of benzene (concentration: 0.1%, benzene: 0.5 g) was introduced intermittently. After benzene was introduced, fluorine gas was bubbled, and lastly the inside of the reactor was sufficiently replaced with nitrogen gas. The solvent was distilled, and 57 g of Compound 6-3 was obtained.

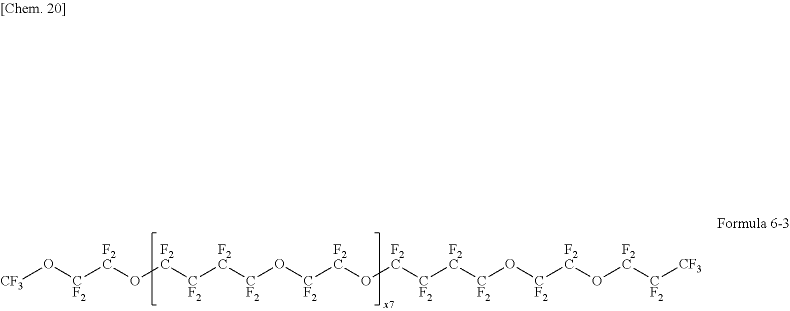

Mean value of x7: 9, Polydispersity index through GPC: 1.36

The NMR spectrum of Compound 6-3;

¹⁹F-NMR (282.7 MHz, solvent: CDCl₃, reference: CFCl₃) δ (ppm): −55 (3F), −81 (3F), −83(22F), −87 (44F), −124 (40F), −129 (2F).

### Example 7

### Example 7-1

Fifty g of CF₂═CFOCF₂CF₂CF₃ and 52.3 g of F₂C═CFOCF₂CF₂CF₂CH₂OH were loaded into a 250-mL metal reactor and stirred at 160° C. The temperature inside the reactor was brought to 25° C., the obtained crude liquid was purified through column chromatography, and 30.7 g of Compound 7-1 was obtained.

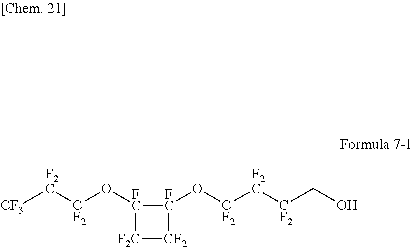

### Example 7-2

Thirty g of Compound 7-1, 13.6 g of CF₂═CF—O—CF₂CF₂CF₂CF₂CF₂CF₂—O—CF═CF₂, and 7.6 g of potassium carbonate were loaded into a 300-mL recovery flask and stirred at 40° C. Hydrochloric acid was added, and an organic phase obtained through separation was dehydrated with magnesium sulfate. Magnesium sulfate was filtered out, the crude liquid was concentrated, and 42.7 g of Compound 7-2 was obtained.

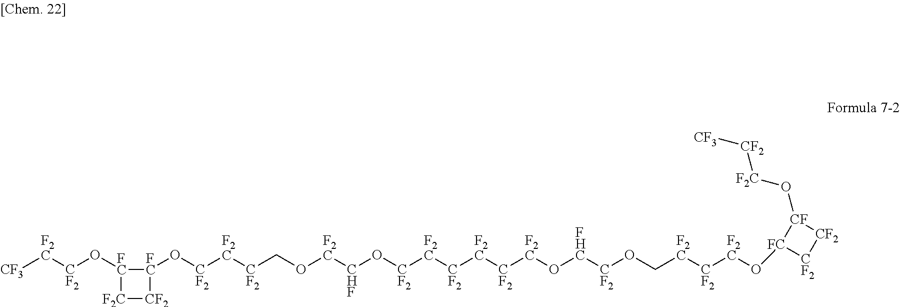

The NMR spectrum of Compound 7-2;

¹H-NMR (300.4 MHz, solvent: CDCl₃, reference: TMS) δ (ppm): 6.0 (2H), 4.6 (4H).

¹⁹F-NMR (282.7 MHz, solvent: CDCl₃, reference: CFCl₃) δ (ppm): −81 (6F), −83 (8F), −85 (4F), −91 (4F), −120 (4F), −124 (4F), −126 (4F), −127 (4F), −128 (4F), −129 (4F), −131 (4F), −137 (2F), −139 (2F), −144 (2F).

### Example 7-3

Two hundred fifty mL of CFE-419 was loaded into a 500-mL metal reactor, and after nitrogen gas was bubbled, fluorine gas of 20 volume % diluted with nitrogen gas was bubbled. A CFE-419 solution of Compound 7-2 (concentration: 30%, Compound 7-2: 40 g) was introduced thereinto. The ratio of the introduction rate (mol/hour) of fluorine gas and the introduction rate (mol/hour) of hydrogen atoms in Compound 7-2 was controlled to 2:1. After Compound 7-2 was introduced, a CFE-419 solution of benzene (concentration: 0.1%, benzene: 0.1 g) was introduced intermittently. After benzene was introduced, fluorine gas was bubbled, and lastly the inside of the reactor was sufficiently replaced with nitrogen gas. The solvent was distilled, and 41 g of Compound 7-3 was obtained.

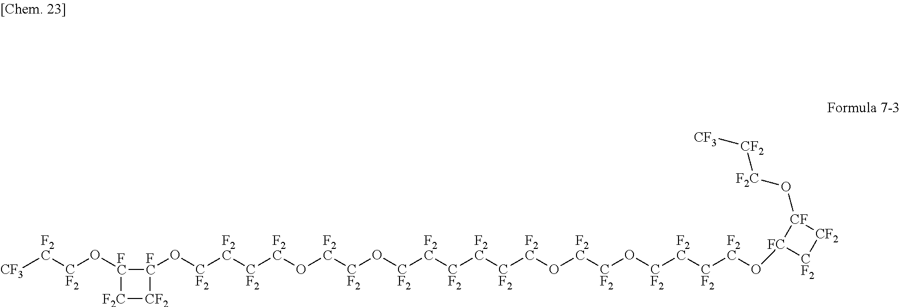

The NMR spectrum of Compound 7-3;

¹⁹F-NMR (282.7 MHz, solvent: CDCl₃, reference: CFCl₃) δ (ppm): −81 (6F), −83 (16F), −87 (8F), −124 (12F), −127 (4F), −128 (4F), −129 (4F), −131 (4F), −137 (2F), −139 (2F).

### Example 8

### Example 8-1

Fifty g of CF₂═CF—O—CF(CF₃)CF₂—O—CF₂CF₂CF₃ and 32.2 g of F₂C═CFOCF₂CF₂CF₂CH₂OH were loaded into a 250-mL metal reactor and stirred at 160° C. The temperature inside the reactor was brought to 25° C., the obtained crude liquid was purified through column chromatography, and 24.7 g of Compound 8-1 was obtained.

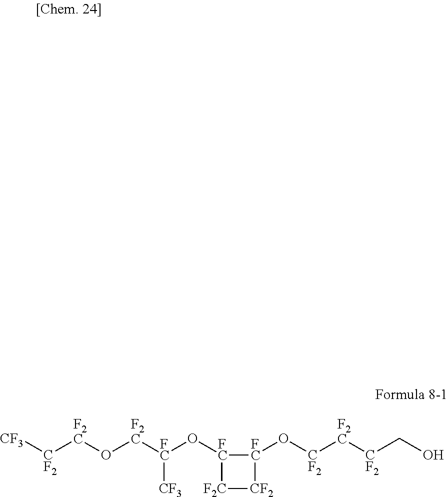

The NMR spectrum of Compound 8-1;

¹H-NMR (300.4 MHz, solvent: CDCl₃, reference: TMS) δ (ppm): 4.0 (2H).

¹⁹F-NMR (282.7 MHz, solvent: CDCl₃, reference: CFCl₃) δ (ppm): −81 (3F), −83 (5F), −85 (2F), −87 (2F), −120 (2F), −126 (2F), −128 (2F), −129 (2F), −131 (3F), −137 (1F), −139 (1F).

### Example 8-2

Twenty g of Compound 8-1, 7.0 g of CF₂═CF—O—CF₂CF₂CF₂CF₂CF₂CF₂—O—CF═CF₂, and 3.9 g of potassium carbonate were loaded into a 300-mL recovery flask and stirred at 40° C. Hydrochloric acid was added, and an organic phase obtained through separation was dehydrated with magnesium sulfate. Magnesium sulfate was filtered out, the crude liquid was concentrated, and 26.4 g of Compound 8-2 was obtained.

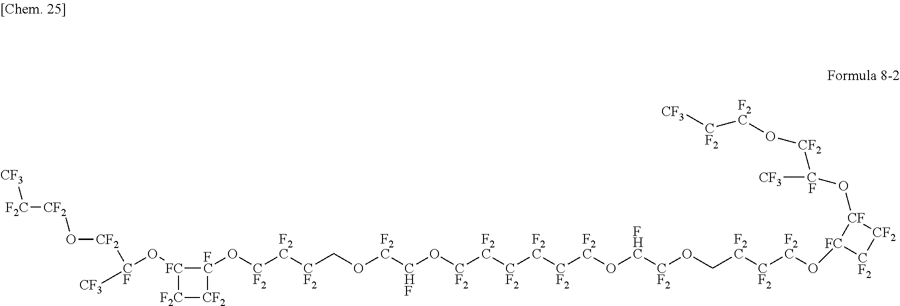

The NMR spectrum of Compound 8-2;

¹H-NMR (300.4 MHz, solvent: CDCl₃, reference: TMS) δ (ppm): 6.0 (2H), 4.6 (4H).

¹⁹F-NMR (282.7 MHz, solvent: CDCl₃, reference: CFCl₃) δ (ppm): −81 (6F), −83 (14F), −85 (4F), −87 (4F), −91 (4F), −120 (4F), −124 (4F), −126 (4F), −127 (4F), −128 (4F), −129 (4F), −131 (6F), −137 (2F), −139 (2F), −144 (2F).

### Example 8-3

Two hundred fifty mL of CFE-419 was loaded into a 500-mL metal reactor, and after nitrogen gas was bubbled, fluorine gas of 20 volume % diluted with nitrogen gas was bubbled. A CFE-419 solution of Compound 8-2 (concentration: 30%, Compound 8-2: 25 g) was introduced thereinto. The ratio of the introduction rate (mol/hour) of fluorine gas and the introduction rate (mol/hour) of hydrogen atoms in Compound 8-2 was controlled to 2:1. After Compound 8-2 was introduced, a CFE-419 solution of benzene (concentration: 0.1%, benzene: 0.1 g) was introduced intermittently. After benzene was introduced, fluorine gas was bubbled, and lastly the inside of the reactor was sufficiently replaced with nitrogen gas. The solvent was distilled, and 26 g of Compound 8-3 was obtained.

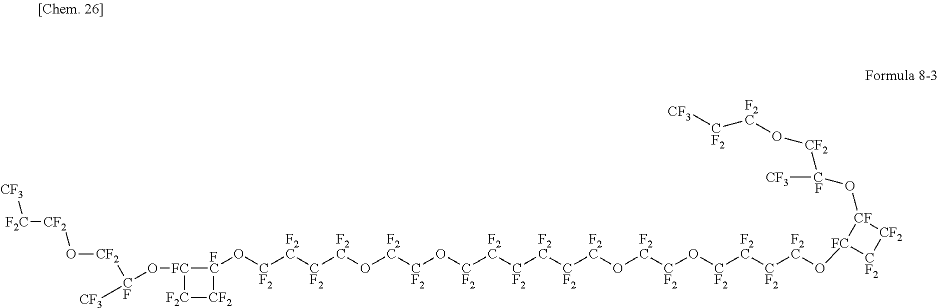

The NMR spectrum of Compound 8-3;

¹⁹F-NMR (282.7 MHz, solvent: CDCl₃, reference: CFCl₃) δ (ppm): −81 (6F), −83 (22F), −87 (12F), −124 (12F), −127 (4F), −128 (4F), −129 (4F), −131 (6F), −137 (2F), −139 (2F).

### Example 9

### Example 9-1

Fifty g of CF₂═CF—O—CF₂CF₂CF₃, 16.9 g of 1,4-butanediol, and 25.9 g of potassium carbonate were loaded into a 300-mL recovery flask and stirred at 40° C. Hydrochloric acid was added, and an organic phase obtained through separation was dehydrated with magnesium sulfate. Magnesium sulfate was filtered out, the crude liquid was concentrated, and 20.1 g of Compound 9-1 was obtained.

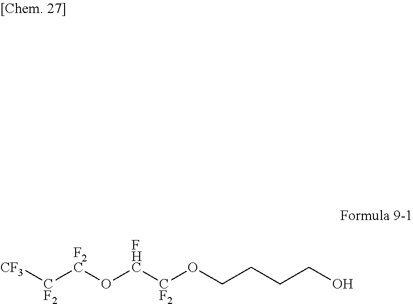

### Example 9-2

Twenty g of Compound 9-1, 13.9 g of CF₂═CF—O—CF₂CF₂CF₂CF₂CF₂CF₂—O—CF═CF₂, and 7.7 g of potassium carbonate were loaded into a 300-mL recovery flask and stirred at 40° C. Hydrochloric acid was added, and an organic phase obtained through separation was dehydrated with magnesium sulfate. Magnesium sulfate was filtered out, the crude liquid was concentrated, and 33.2 g of Compound 9-2 was obtained.

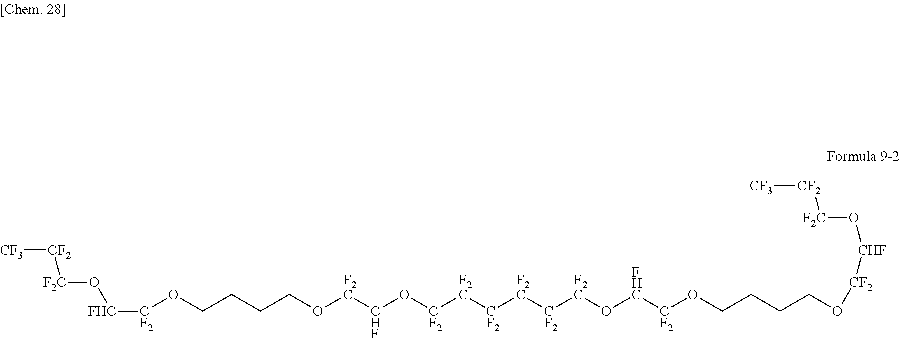

The NMR spectrum of Compound 9-2;

¹H-NMR (300.4 MHz, solvent: CDCl₃, reference: TMS) δ (ppm): 6.0 (4H), 3.5 (8H), 1.7 (8H).

¹⁹F-NMR (282.7 MHz, solvent: CDCl₃, reference: CFCl₃) δ (ppm): −81 (6F), −83 (4F), −85 (4F), −91 (8F), −124 (4F), −127 (4F), −129 (4F), −144 (4F).

### Example 9-3

Two hundred fifty mL of CFE-419 was loaded into a 500-mL metal reactor, and after nitrogen gas was bubbled, fluorine gas of 20 volume % diluted with nitrogen gas was bubbled. A CFE-419 solution of Compound 9-2 (concentration: 30%, Compound 9-2: 30 g) was introduced thereinto. The ratio of the introduction rate (mol/hour) of fluorine gas and the introduction rate (mol/hour) of hydrogen atoms in Compound 9-2 was controlled to 2:1. After Compound 9-2 was introduced, a CFE-419 solution of benzene (concentration: 0.1%, benzene: 0.1 g) was introduced intermittently. After benzene was introduced, fluorine gas was bubbled, and lastly the inside of the reactor was sufficiently replaced with nitrogen gas. The solvent was distilled, and 38 g of Compound 9-3 was obtained.

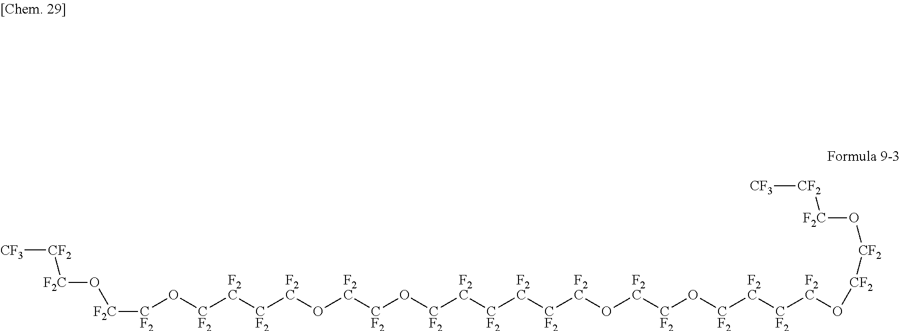 [SMILES: FC(F)(F)C(F)(F)C(F)(F)OC(F)(F)C(F)(F)OC(F)(F)C(F)(F)C(F)(F)C(F)(F)OC(F)(F)C(F)(F)OC(F)(F)C(F)(F)C(F)(F)C(F)(F)C(F)(F)C(F)(F)OC(F)(F)C(F)(F)OC(F)(F)C(F)(F)C(F)(F)C(F)(F)OC(F)(F)C(F)(F)OC(F)(F)C(F)(F)C(F)(F)F]

The NMR spectrum of Compound 9-3;

¹⁹F-NMR (282.7 MHz, solvent: CDCl₃, reference: CFCl₃) δ (ppm): −81 (6F), −83 (16F), −87 (16F), −124 (12F), −127 (4F), −129 (4F).

### Example 10

### Example 10-1

Fifty g of CF₂═CF—O—CF(CF₃)CF₂—O—CF₂CF₂CF₃, 10.4 g of 1,4-butanediol, and 16.0 g of potassium carbonate were loaded into a 300-mL recovery flask and stirred at 40° C. Hydrochloric acid was added, and an organic phase obtained through separation was dehydrated with magnesium sulfate. Magnesium sulfate was filtered out, the crude liquid was concentrated, and 18.1 g of Compound 10-1 was obtained.

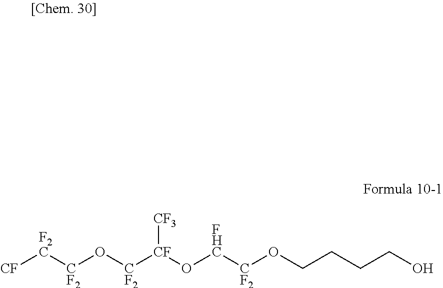

### Example 10-2

Eighteen g of Compound 10-1, 8.5 g of CF₂═CF—O—CF₂CF₂CF₂CF₂CF₂CF₂—O—CF═CF₂, and 4.8 g of potassium carbonate were loaded into a 300-mL recovery flask and stirred at 40° C. Hydrochloric acid was added, and an organic phase obtained through separation was dehydrated with magnesium sulfate. Magnesium sulfate was filtered out, the crude liquid was concentrated, and 26.0 g of Compound 10-2 was obtained.

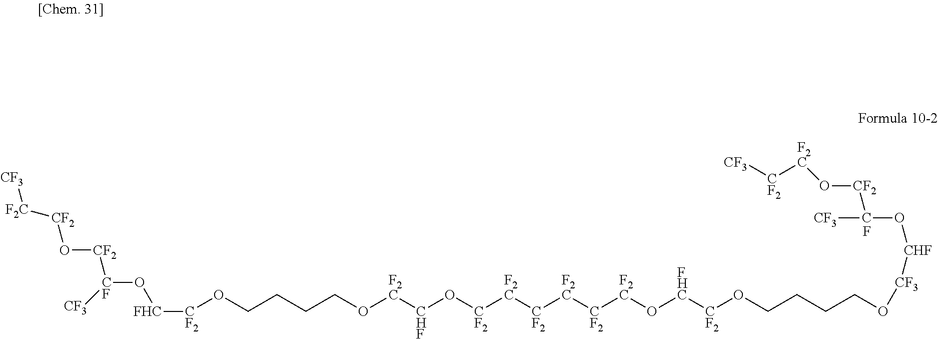

The NMR spectrum of Compound 10-2;

¹H-NMR (300.4 MHz, solvent: CDCl₃, reference: TMS) δ (ppm): 6.0 (4H), 3.5 (8H), 1.7 (8H).

¹⁹F-NMR (282.7 MHz, solvent: CDCl₃, reference: CFCl₃) δ (ppm): −81 (6F), −83 (14F), −87 (4F), −91 (8F), −124 (4F), −127 (4F), −129 (4F), −131 (2F), −144 (4F).

### Example 10-3

Two hundred fifty mL of CFE-419 was loaded into a 500-mL metal reactor, and after nitrogen gas was bubbled, fluorine gas of 20 volume % diluted with nitrogen gas was bubbled. A CFE-419 solution of Compound 10-2 (concentration: 30%, Compound 10-2: 25 g) was introduced thereinto. The ratio of the introduction rate (mol/hour) of fluorine gas and the introduction rate (mol/hour) of hydrogen atoms in Compound 10-2 was controlled to 2:1. After Compound 10-2 was introduced, a CFE-419 solution of benzene (concentration: 0.1%, benzene: 0.1 g) was introduced intermittently. After benzene was introduced, fluorine gas was bubbled, and lastly the inside of the reactor was sufficiently replaced with nitrogen gas. The solvent was distilled, and 30 g of Compound 10-3 was obtained.

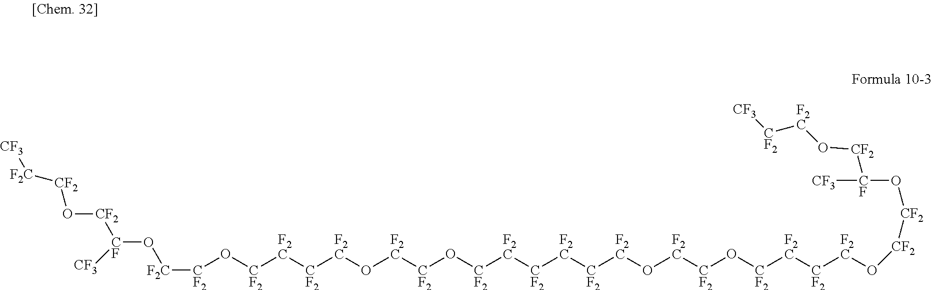

The NMR spectrum of Compound 10-3;

¹⁹F-NMR (282.7 MHz, solvent: CDCl₃, reference: CFCl₃) δ (ppm): −81 (6F), −83 (22F), −87 (20F), −124 (12F), −127 (4F), −129 (4F), −131 (2F).

### Example 11

### Example 11-1

Fifty g of 1,2-benzenediol, 3,400 g of CF₂═CF—O—CF₂CF₂CF₂CF₂—O—CF═CF₂, and 62.7 g of potassium carbonate were loaded into a 3,000-mL recovery flask and stirred at 40° C. Hydrochloric acid was added, and an organic phase obtained through separation was dehydrated with magnesium sulfate. Magnesium sulfate was filtered out, the crude liquid was concentrated, and 199.9 g of Compound 11-1 was obtained.

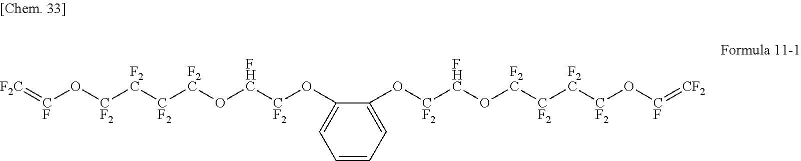

The NMR spectrum of Compound 11-1;

¹H-NMR (300.4 MHz, solvent: CDCl₃, reference: TMS) δ (ppm): 6.9 (2H), 6.2 (2H), 6.0 (2H).

¹⁹F-NMR (282.7 MHz, solvent: CDCl₃, reference: CFCl₃) δ (ppm): −83 (8F), −91 (4F), −116 (2F), −124 (10F), −137 (2F), −144 (2F).

### Example 11-2

One thousand mL of CFE-419 was loaded into a 3,000-mL metal reactor, and after nitrogen gas was bubbled, fluorine gas of 20 volume % diluted with nitrogen gas was bubbled. A CFE-419 solution of Compound 11-1 (concentration: 30%, Compound 11-1: 190 g) was introduced thereinto. The ratio of the introduction rate (mol/hour) of fluorine gas and the introduction rate (mol/hour) of hydrogen atoms in Compound 11-1 was controlled to 2:1. After Compound 11-1 was introduced, a CFE-419 solution of benzene (concentration: 0.1%, benzene: 0.1 g) was introduced intermittently.

After benzene was introduced, fluorine gas was bubbled, and lastly the inside of the reactor was sufficiently replaced with nitrogen gas. The solvent was distilled, and 243 g of Compound 11-2 was obtained.

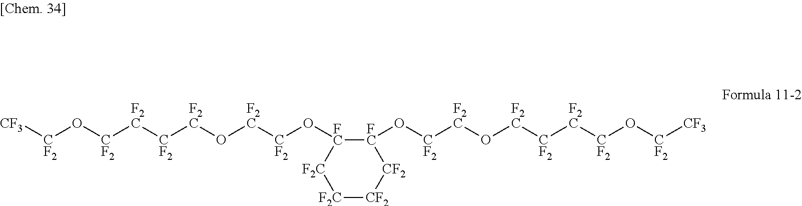

The NMR spectrum of Compound 11-2;

¹⁹F-NMR (282.7 MHz, solvent: CDCl₃, reference: CFCl₃) δ (ppm): −83 (12F), −87 (14F), −124 (8F), −127 (4F), −131 (4F), −190 (2F).

### Example 12

### Example 12-1

191.7 g of Compound 12-1 was obtained in a manner similar to that in Example 11-1, except that 50 g of 1,3-benzenediol was used in place of 50 g of 1,2-benzenediol in Example 11-1.

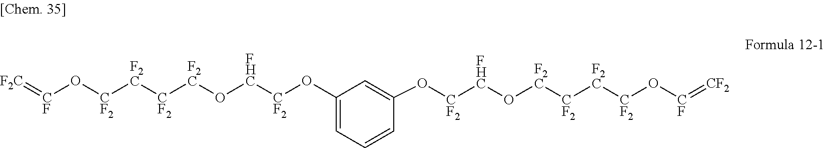

The NMR spectrum of Compound 12-1;

¹H-NMR (300.4 MHz, solvent: CDCl₃, reference: TMS) δ (ppm): 7.1 (1H), 6.6 (2H), 6.5 (1H), 6.0 (2H).

¹⁹F-NMR (282.7 MHz, solvent: CDCl₃, reference: CFCl₃) δ (ppm): −83 (8F), −91 (4F), −116 (2F), −124 (10F), −137 (2F), −144 (2F).

### Example 12-2

Two hundred forty-three g of Compound 12-2 was obtained in a manner similar to that in Example 11-2, except that 190 g of Compound 12-1 was used in place of 190 g of Compound 11-1 in Example 11-2.

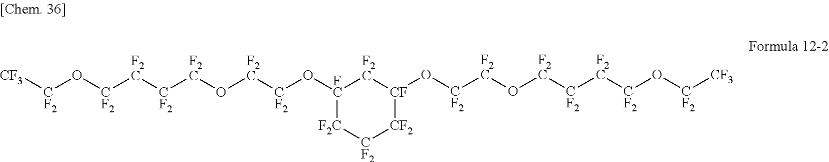

The NMR spectrum of Compound 12-2;

¹⁹F-NMR (282.7 MHz, solvent: CDCl₃, reference: CFCl₃) δ (ppm): −83 (12F), −87 (14F), −124 (8F), −127 (6F), −131 (2F), −190 (2F).

### Example 13

### Example 13-1

Fifty g of phthalyl alcohol, 2,709 g of CF₂═CF—O—CF₂CF₂CF₂CF₂—O—CF═CF₂, and 49.9 g of potassium carbonate were loaded into a 3,000-mL recovery flask and stirred at 40° C. Hydrochloric acid was added, and an organic phase obtained through separation was dehydrated with magnesium sulfate. Magnesium sulfate was filtered out, the crude liquid was concentrated, and 157.5 g of Compound 13-1 was obtained.

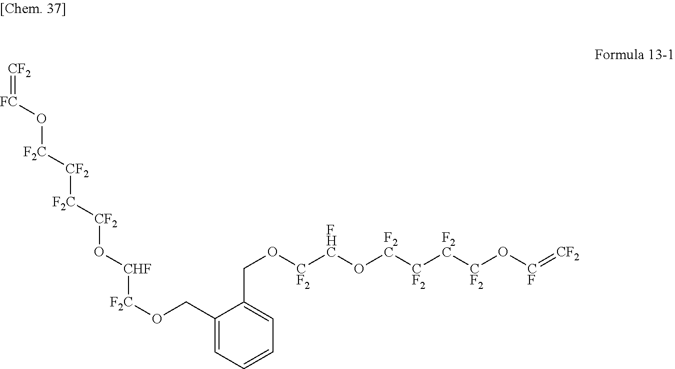

The NMR spectrum of Compound 13-1;

¹H-NMR (300.4 MHz, solvent: CDCl₃, reference: TMS) δ (ppm): 7.3 (2H), 7.1 (2H), 6.0 (2H), 4.6 (4H).

¹⁹F-NMR (282.7 MHz, solvent: CDCl₃, reference: CFCl₃) δ (ppm): −83 (8F), −91 (4F), −116 (2F), −124 (10F), −137 (2F), −144 (2F).

### Example 13-2

One thousand mL of CFE-419 was loaded into a 3,000-mL metal reactor, and after nitrogen gas was bubbled, fluorine gas of 20 volume % diluted with nitrogen gas was bubbled. A CFE-419 solution of Compound 13-1 (concentration: 30%, Compound 13-1: 150 g) was introduced thereinto. The ratio of the introduction rate (mol/hour) of fluorine gas and the introduction rate (mol/hour) of hydrogen atoms in Compound 13-1 was controlled to 2:1. After Compound 13-1 was introduced, a CFE-419 solution of benzene (concentration: 0.1%, benzene: 0.1 g) was introduced intermittently. After benzene was introduced, fluorine gas was bubbled, and lastly the inside of the reactor was sufficiently replaced with nitrogen gas. The solvent was distilled, and 187 g of Compound 13-2 was obtained.

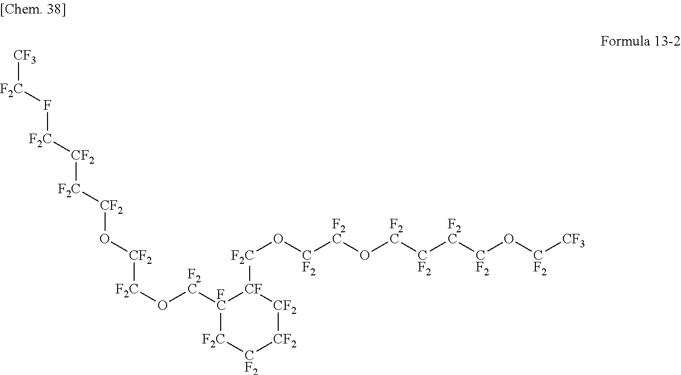

The NMR spectrum of Compound 13-2;

¹⁹F-NMR (282.7 MHz, solvent: CDCl₃, reference: CFCl₃) δ (ppm): −83 (12F), −87 (18F), −124 (8F), −127 (4F), −131 (4F), −184 (2F).

### Example 14

### Example 14-1

Fifty g of 1,4-benzenediol, 3,400 g of CF₂═CF—O—CF₂CF₂CF₂CF₂—O—CF═CF₂, and 62.7 g of potassium carbonate were loaded into a 3,000-mL recovery flask and stirred at 40° C. Hydrochloric acid was added, and an organic phase obtained through separation was dehydrated with magnesium sulfate. Magnesium sulfate was filtered out, the crude liquid was concentrated, and 191.7 g of Compound 14-1 was obtained.

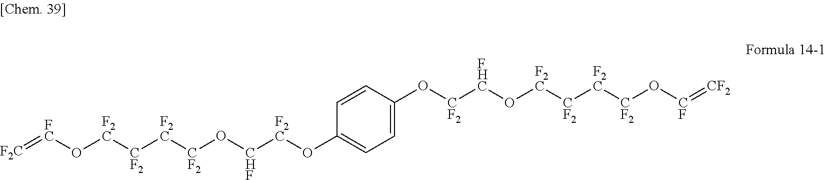

The NMR spectrum of Compound 14-1;

¹H-NMR (300.4 MHz, solvent: CDCl₃, reference: TMS) δ (ppm): 6.9 (4H), 6.0(2H).

¹⁹F-NMR (282.7 MHz, solvent: CDCl₃, reference: CFCl₃) δ (ppm): −83 (8F), −91 (4F), −116 (2F), −124 (10F), −137 (2F), −144 (2F).

### Example 14-2

One thousand mL of CFE-419 was loaded into a 3,000-mL metal reactor, and after nitrogen gas was bubbled, fluorine gas of 20 volume % diluted with nitrogen gas was bubbled. A CFE-419 solution of Compound 14-1 (concentration: 30%, Compound 14-1: 190 g) was introduced thereinto. The ratio of the introduction rate (mol/hour) of fluorine gas and the introduction rate (mol/hour) of hydrogen atoms in Compound 14-1 was controlled to 2:1. After Compound 14-1 was introduced, a CFE-419 solution of benzene (concentration: 0.1%, benzene: 0.1 g) was introduced intermittently. After benzene was introduced, fluorine gas was bubbled, and lastly the inside of the reactor was sufficiently replaced with nitrogen gas. The solvent was distilled, and 243 g of Compound 14-2 was obtained.

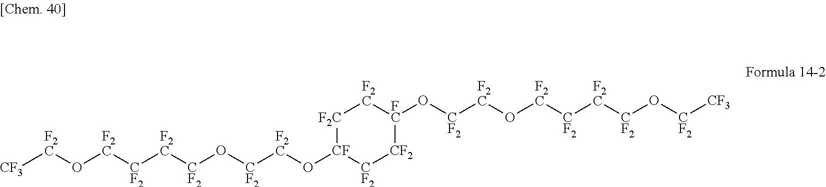

The NMR spectrum of Compound 14-2;

¹⁹F-NMR (282.7 MHz, solvent: CDCl₃, reference: CFCl₃) δ (ppm): −83 (12F), −87 (14F), −124 (8F), −127 (8F), −190 (2F).

### Example 15

### Example 15-1

Fifty g of p-xylene glycol, 2,709 g of CF₂═CF—O—CF₂CF₂CF₂CF₂—O—CF═CF₂, and 49.9 g of potassium carbonate were loaded into a 3,000-mL recovery flask and stirred at 40° C. Hydrochloric acid was added, and an organic phase obtained through separation was dehydrated with magnesium sulfate. Magnesium sulfate was filtered out, the crude liquid was concentrated, and 157.5 g of Compound 15-1 was obtained.

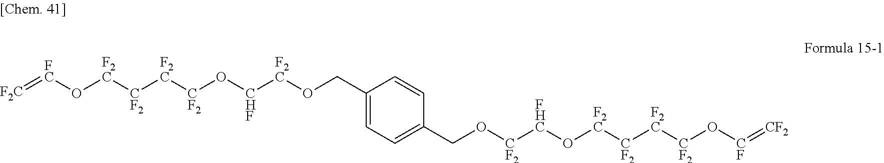

The NMR spectrum of Compound 15-1;

¹H-NMR (300.4 MHz, solvent: CDCl₃, reference: TMS) δ (ppm): 6.9 (4H), 6.0 (2H), 4.7 (4H).

¹⁹F-NMR (282.7 MHz, solvent: CDCl₃, reference: CFCl₃) δ (ppm): −83 (8F), −91 (4F), −116 (2F), −124 (10F), −137 (2F), −144 (2F).

### Example 15-2

One thousand mL of CFE-419 was loaded into a 3,000-mL metal reactor, and after nitrogen gas was bubbled, fluorine gas of 20 volume % diluted with nitrogen gas was bubbled. A CFE-419 solution of Compound 15-1 (concentration: 30%, Compound 15-1: 150 g) was introduced thereinto. The ratio of the introduction rate (mol/hour) of fluorine gas and the introduction rate (mol/hour) of hydrogen atoms in Compound 15-1 was controlled to 2:1. After Compound 15-1 was introduced, a CFE-419 solution of benzene (concentration: 0.1%, benzene: 0.1 g) was introduced intermittently. After benzene was introduced, fluorine gas was bubbled, and lastly the inside of the reactor was sufficiently replaced with nitrogen gas. The solvent was distilled, and 187 g of Compound 15-2 was obtained.

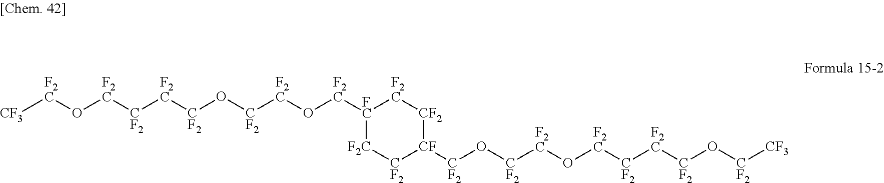

The NMR spectrum of Compound 15-2;

¹⁹F-NMR (282.7 MHz, solvent: CDCl₃, reference: CFCl₃) δ (ppm): −83 (12F), −87 (18F), −124 (8F), −127 (8F), −184 (2F).

### Example 16

### Example 16-1

Twenty g of Compound 3-3 obtained in Example 3-3, 7.1 g of sodium fluoride powder, 20 g of AC-2000, and 20 g of CF₃CF₂CF₂OCF(CF₃)COF were loaded into a 50-mL recovery flask. The above was stirred for 24 hours at 50° C. in nitrogen atmosphere. The temperature inside the recovery flask was brought to 25° C., and then sodium fluoride powder was removed through filtration. Excess CF₃CF₂CF₂OCF(CF₃)COF and AC-2000 were distilled under reduced pressure, and 24 g of Compound 16-1 was obtained.

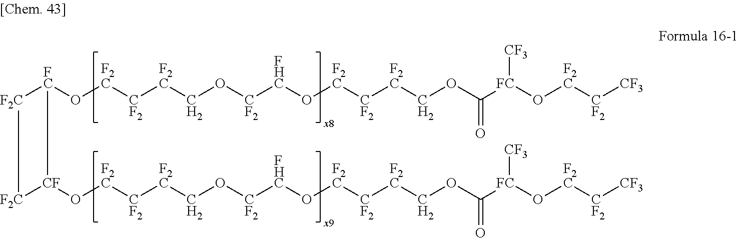

Mean value of x8+x9: 8

The NMR spectrum of Compound 16-1;

¹H-NMR (300.4 MHz, solvent: CDCl₃, reference: TMS) δ (ppm): 6.0 (8H), 5.0 (4H), 4.6 (16H).

¹⁹F-NMR (282.7 MHz, solvent: CDCl₃, reference: CFCl₃) δ (ppm): −79 (4F), −80 (2F), −81 (6F), −82 (6F), −85 (18F), −91 (16F), −119 (4F), −120 (16F), −126 (20F), −128 (2F), −129 (4F), −131 (2F), −131 (2F), −137 (1F), −139 (1F), −144 (8F).

### Example 16-2

Two hundred fifty mL of CFE-419 was loaded into a 500-mL metal reactor, and after nitrogen gas was bubbled, fluorine gas of 20 volume % diluted with nitrogen gas was bubbled. A CFE-419 solution of Compound 16-1 obtained in Example 16-1 (concentration: 10%, Compound 16-1: 20 g) was introduced thereinto over 3 hours. The ratio of the introduction rate (mol/hour) of fluorine gas and the introduction rate (mol/hour) of hydrogen atoms in Compound 16-1 was controlled to 2:1. After introduction of Compound 16-1 was completed, a CFE-419 solution of benzene (concentration: 0.1%, benzene: 0.1 g) was introduced intermittently. After introduction of benzene was completed, fluorine gas was bubbled, and lastly the inside of the reactor was sufficiently replaced with nitrogen gas. The solvent was distilled, and 21 g of Compound 16-2 was obtained.

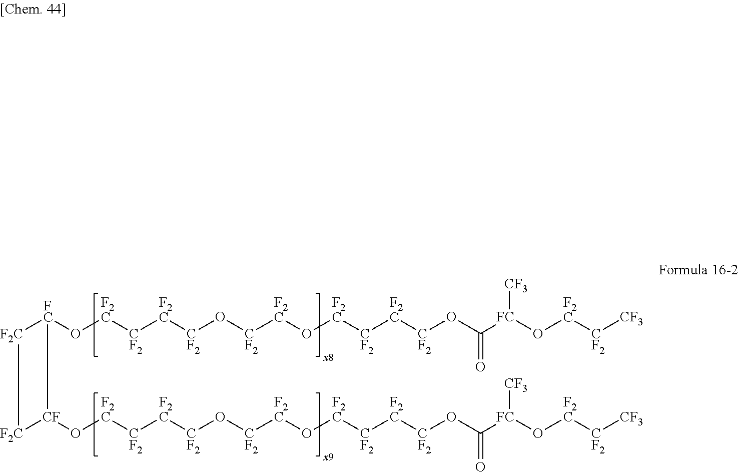

Mean value of x8+x9: 8

The NMR spectrum of Compound 16-2;

¹⁹F-NMR (282.7 MHz, solvent: CDCl₃, reference: CFCl₃) δ (ppm): −79 (4F), −80 (2F), −81 (6F), −82 (6F), −83 (38F), −87 (32F), −124 (40F), −128 (2F), −129 (4F), −131 (2F), −131 (2F), −137 (1F), −139 (1F).

### Example 16-3

Twenty g of Compound 16-2 obtained in Example 16-2, 1.8 g of sodium fluoride, and 20 mL of AC-2000 were loaded into a 50-mL recovery flask and stirred in ice bath. Then, 1.4 g of methanol was introduced and stirred for 1 hour at 25° C. After filtration, the filtrate was purified through column chromatography. Fourteen g of Compound 16-3 was obtained.

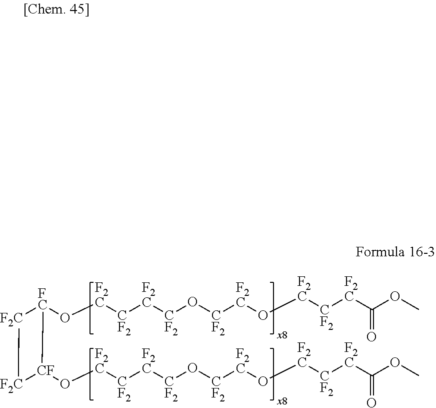

Mean value of x8+x9: 6

The NMR spectrum of Compound 16-3;

¹H-NMR (300.4 MHz, solvent: CDCl₃, reference: TMS) δ (ppm): 4.2 (6H).

¹⁹F-NMR (282.7 MHz, solvent: CDCl₃, reference: CFCl₃) δ (ppm): −80 (2F), −83 (34F), −87 (32F), −119 (4F), −124 (36F), −128 (2F), −131 (2F), −137 (1F), −139 (1F).

### Example 16-4

Six g of Compound 16-3 obtained in Example 16-3, 0.4 g of H₂NCH₂CH═CH₂, and 6 mL of AC-2000 were loaded into a 50-mL recovery flask and stirred for 24 hours at 0° C. The reaction crude liquid was purified through column chromatography. Thus, 3.8 g of Compound 16-4 was obtained.

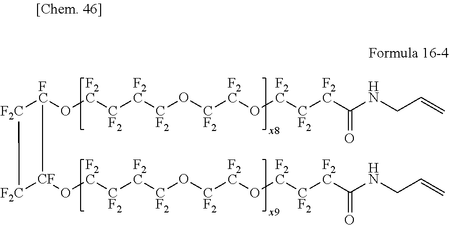

Mean value of x8+x9: 6

The NMR spectrum of Compound 16-4;

¹H-NMR (300.4 MHz, solvent: CDCl₃, reference: TMS) δ (ppm): 9.3 (2H), 5.8 (2H), 5.2 (2H), 5.1 (2H), 4.3 (4H).

¹⁹F-NMR (282.7 MHz, solvent: CDCl₃, reference: CFCl₃) δ (ppm): −80 (2F), −83 (34F), −87 (32F), −117 (4F), −124 (36F), −128 (2F), −131 (2F), −137 (1F), −139 (1F).

### Example 17

Six g of Compound 16-3 obtained in Example 16-3, 0.4 g of H₂NCH(CH₃)₂, and 6 mL of AC-2000 were loaded into a 50-mL recovery flask and stirred for 24 hours at 0° C. The reaction crude liquid was purified through column chromatography. Thus, 4.0 g of Compound 17-1 was obtained.

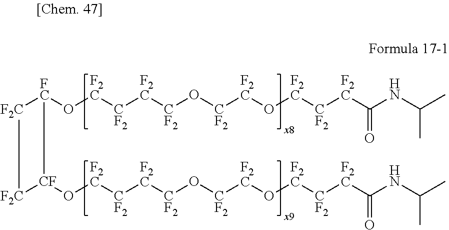

Mean value of x8+x9: 6

The NMR spectrum of Compound 17-1;

¹H-NMR (300.4 MHz, solvent: CDCl₃, reference: TMS) δ (ppm): 8.1 (2H), 3.8 (2H), 1.0 (12H).

¹⁹F-NMR (282.7 MHz, solvent: CDCl₃, reference: CFCl₃) δ (ppm): −80 (2F), −83 (34F), −87 (32F), −117 (4F), −124 (36F), −128 (2F), −131 (2F), −137 (1F), −139 (1F).

### Example 18

Eight g of Compound 16-3 obtained in Example 16-3, 1.6 g of HN(CH₂CH₃)₂, and 6 mL of AC-2000 were loaded into a 50-mL recovery flask and stirred for 24 hours at 0° C. The reaction crude liquid was purified through column chromatography. Thus, 3.2 g of Compound 18-1 was obtained.

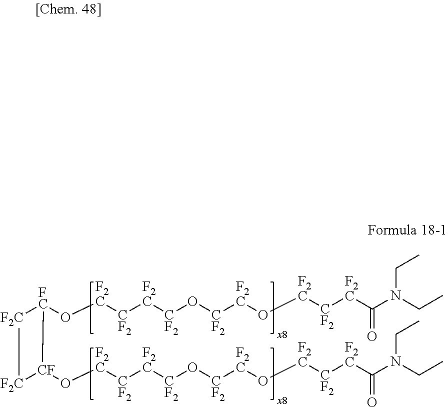

Mean value of x8+x9: 6

The NMR spectrum of Compound 18-1;

¹H-NMR (300.4 MHz, solvent: CDCl₃, reference: TMS) δ (ppm): 3.3 (8H), 1.1 (12H).

¹⁹F-NMR (282.7 MHz, solvent: CDCl₃, reference: CFCl₃) δ (ppm): −80 (2F), −83 (34F), −87 (32F), −110 (4F), −124 (36F), −128 (2F), −131 (2F), −137 (1F), −139 (1F).

### [Evaluation]

### <Evaluation of Weight Loss Rate>

Each of the fluorine-containing ether compounds obtained in Examples 1 to 18 was measured under the following condition with use of a thermogravimetry/differential thermal analyzer (TG/DTA), and the heat resistance was evaluated against the following evaluation criteria based on the temperature held when the weight loss rate reached 50%. The result is shown in Table 1. Table 1 also shows the molecular weight (or the number-average molecular weight) of each fluorine-containing ether compound.

### (Device Condition)

Measurement Start Temperature: 25° C.

Maximum Temperature: 500° C.

Rate of Heating: 10° C./min

### (Evaluation Criteria)

S: Temperature held at 50% weight loss was no lower than 230° C.

A: Temperature held at 50% weight loss was no lower than 220° C. but lower than 230° C.

B: Temperature held at 50% weight loss was no lower than 215° C. but lower than 220° C.

C: Temperature held at 50% weight loss was no lower than 210° C. but lower than 215° C.

D: Temperature held at 50% weight loss was no lower than 200° C. but lower than 210° C.

E: Temperature held at 50% weight loss was lower than 200° C.

TABLE 1

| | | | | Molecular |
|---|---|---|---|---|
| | | | | Weight or |
| | | | Temperature | Number- |
| | | Evaluation | at 50% | Average |
| | | of Weight | Weight Loss | Molecular |
| Examples | Compounds | Loss Rate | (° C.) | Weight |
| Example 1 | Compound 1-3 | S | 240 | 4000 |
| Example 2 | Compound 2-2 | S | 243 | 4000 |
| Example 3 | Compound 3-5 | S | 231 | 4000 |
| Example 4 | Compound 4-2 | C | 214 | 1196 |
| Example 7 | Compound 7-3 | B | 215 | 1690 |
| Example 8 | Compound 8-3 | A | 221 | 2022 |
| Example 11 | Compound 11-2 | C | 212 | 1196 |
| Example 12 | Compound 12-2 | C | 213 | 1196 |
| Example 13 | Compound 13-2 | C | 214 | 1296 |
| Example 14 | Compound 14-2 | C | 211 | 1196 |
| Example 15 | Compound 15-2 | C | 210 | 1296 |
| Example 16 | Compound 16-4 | S | 254 | 4000 |
| Example 17 | Compound 17-1 | S | 268 | 4000 |
| Example 18 | Compound 18-1 | S | 260 | 4000 |
| Example 5 | Compound 5-1 | D | 205 | 4000 |
| Example 6 | Compound 6-3 | D | 209 | 4000 |
| Example 9 | Compound 9-3 | E | 186 | 1566 |
| Example 10 | Compound 10-3 | E | 198 | 1898 |

This result shows that the present compound that includes a carbocycle having no heteroatom and a fluoroalkyl group or an amide group excels in heat resistance.

### <Manufacturing and Evaluation of Article>

A surface of a substrate was treated through a dry coating technique or a wet coating technique described below with use of a mixture in which each of the fluorine-containing ether compounds shown in Table 2 and Compound 1-3a-1 fabricated through the following method were mixed at a mass ratio of 1:1, and thus each substrate having a surface treatment layer was manufactured. Chemically strengthened glass was used for a substrate. Each obtained substrate having a surface treatment layer was evaluated through the following method. The result is shown in Table 2.

(Method of Fabricating Compound 1-3a-1)

Compound 1-3a-1 was obtained in accordance with the description of Example 1-6 of Examples in International Publication No. WO2013/121984. In Formula (1-3a-1), the mean value of n is 7.

CF₃CF₂—O—(CF₂CF₂O—CF₂CF₂CF₂CF₂O)ₙ—CF₂CF₂OCF₂CF₂CF₂C(═O)NHCH₂CH₂CH₂—Si(OCH₃)₃ Formula (1-3a-1)

### (Dry Coating Technique)

Dry coating was done with use of a vacuum deposition device (manufactured by ULVAC, Inc., product name: VTR-350M) (vacuum deposition technique). A molybdenum boat inside the vacuum deposition device was charged with 0.5 g of each of the mixtures of the corresponding fluorine-containing ether compounds, and the inside of the vacuum deposition device was exhausted to no higher than 1×10⁻³ Pa. The boat having a mixture placed thereon was heated at a rate of heating of no higher than 10° C./minute, the shutter was opened when the rate of deposition exceeded 1 nm/second as measured by a quartz-crystal oscillator thickness gauge to start deposition on the surface of a substrate. The shutter was closed when the film thickness reached about 50 nm to end the deposition onto the surface of the substrate. The substrate on which the mixture was deposited was subjected to heat treatment for 30 minutes at 200° C. Then, the substrate was washed with AK-225 (product name, manufactured by AGC Inc.), which is a fluorine-containing solvent, and thus a substrate having a surface treatment layer was obtained.

### (Wet Coating Technique)

Each of the mixtures of the corresponding fluorine-containing ether compounds shown in Table 2 and Novec-7200 (product name, manufactured by 3M) serving as a medium were mixed together to prepare a coating liquid having a solid content concentration of 0.05%. A substrate was dipped into this coating liquid (dip coating technique), left for 30 minutes, and then pulled out. The substrate was dried for 30 minutes at 200° C. and washed with AK-225 (product name, manufactured by AGC Inc.), which is a fluorine-containing solvent, and thus a substrate having a surface treatment layer was obtained.

### (Method of Evaluation)

The dynamic friction coefficient of each surface layer with respect to artificial skin (manufactured by Idemitsu Technofine Co., Ltd., product name: PBZ13001) was measured under the condition in which the contact area was 3 cm by 3 cm and the load was 0.98 N with use of a variable normal load friction and wear measurement system (manufactured by Shinto Scientific Co., Ltd., product name: HHS2000). The higher the dynamic friction coefficient, the higher the slip resistance.

Good: The dynamic friction coefficient is no smaller than 0.5.

Acceptable: The dynamic friction coefficient is no smaller than 0.3 but smaller than 0.5.

Unacceptable: The dynamic friction coefficient is smaller than 0.3.

TABLE 2

| | | | Fluorine- | |
|---|---|---|---|---|
| | | Fluorine- | Containing | |
| | Method | Containing Ether | Ether | Slip |
| | of Coating | Compound 1 | Compound 2 | Resistance |
| Example 19 | Dry | Compound 1-3a-1 | Compound 5-1 | Un- |
| | | | | acceptable |
| Example 20 | Wet | Compound 1-3a-1 | Compound 5-1 | Un- |
| | | | | acceptable |
| Example 21 | Dry | Compound 1-3a-1 | Compound 3-5 | Good |
| Example 22 | Wet | Compound 1-3a-1 | Compound 3-5 | Good |

### INDUSTRIAL APPLICABILITY

A fluorine-containing ether compound according to the present invention can be used in a variety of circumstances where heat resistance, slip resistance, water repellency, or oil repellency is desired. For example, the fluorine-containing ether compound can be used for a display input device, such as a touch panel; a surface protective coat of a member made of transparent glass or transparent plastics, or a soil-resistant coat in kitchen; a water-repellent moisture proof coat or a soil-resistant coat of an electronic device, a heat exchanger, or a battery, or a soil-resistant coat of toiletries; a coat on a member that needs to be liquid-repellent while conducting electricity; a water-repellent, waterproof, or water-sliding coat of a heat exchanger; or a surface low friction coat of a vibrating strainer or inside a cylinder. More specific use examples include a front protective plate, an antireflection plate, a polarizing plate, an antiglare plate of a display, such plates having a surface subjected to antireflection film treatment, various apparatuses having a display input device whose screen is operated by human fingers or hands, such as a touch panel sheet or a touch panel display of an apparatus such as a mobile phone or a portable information terminal, a decorative building material for restrooms, bathrooms, lavatories, or kitchens, a waterproof coat for a wiring board, a water-repellent, waterproof coat of a heat exchanger, a water-repellent coat of a solar cell, a waterproof, water-repellent coat of a printed wiring board, a waterproof, water-repellent coat of an electronic device casing or an electronic component, an insulating property improving coat of a power transmission line, a waterproof, water-repellent coat of various filters, a waterproof coat of an electromagnetic wave absorbing material or a sound absorbing material, a soil-resistant coat for bathrooms, kitchen instruments, or toiletries, a water-repellent, waterproof, water sliding coat of a heat exchanger, a surface low-friction coat of a vibrating strainer or inside a cylinder, or a surface protective coat of a machine component, a vacuum apparatus component, a bearing component, an automobile component, or an industrial tool.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A fluorine-containing ether compound expressed by the following formula (1) or the following formula (2):
Q¹{—(R^{f12})ₘ₂—O—(R^{f11}O)ₘ₁-A¹}ₙ₁ Formula (1)
{A¹-(OR^{f11})ₘ₁—O—(R^{f12})ₘ₂-}ₙ₂Q²-[(R^{f12})ₘ₂—O—(R^{f11}O)ₘ₁—(R^{f12})ₘ₂-Q³]ₚ—(R^{f12})ₘ₂—O—(R^{f11}O)ₘ₁—(R^{f12})ₘ₂-Q²-{(R^{f12})ₘ₂—O—(R^{f11}O)ₘ₁-A¹}ₙ₂ Formula (2),
wherein
Q¹ is a carbocycle having a carbon number k1 that may have a fluorine atom, an alkyl group, or a fluoroalkyl group as a substituent, and k1 is an integer no smaller than 3,
Q² is a carbocycle having a carbon number k2 that may have a fluorine atom, an alkyl group, or a fluoroalkyl group as a substituent, k2 is an integer no smaller than 3, and the plurality of Q² may be identical to or different from each other,
Q³ is a carbocycle having a carbon number k3 that may have a fluorine atom, an alkyl group, or a fluoroalkyl group as a substituent, and k3 is an integer no smaller than 3,
R^{f11} and R^{f12} are each independently a fluoroalkylene group having a carbon number of from 1 to 6, and when there are a plurality of R^{f11} or R^{f12}, the R^{f11} or R^{f12} may each independently be identical to or different from each other,
A¹ is a fluoroalkyl group having a carbon number of from 1 to 20 or —R^{f21}—C(═O)—NR¹R², and when there are a plurality of A¹, the plurality of A¹ may be identical to or different from each other,
R^{f21} is an alkylene group or a fluoroalkylene group, having a carbon number of from 1 to 6,
R¹ and R² are each independently a hydrogen atom, a fluorine atom, an alkyl group that may have a double bond or a fluoroalkyl group that may have a double bond,
n1 is an integer of from 1 to k1,
n2 is an integer of from 0 to (k2-1), and the plurality of n2 may be identical to or different from each other,
m1 is an integer of from 1 to 500, and the plurality of m1 may be identical to or different from each other,
m2 is each independently 0 or 1, and the plurality of m2 may be identical to or different from each other, and
p is an integer of from 0 to 100.

2. The fluorine-containing ether compound according to claim 1, wherein at least one of the plurality of m2 is 0.

3. The fluorine-containing ether compound according to claim 1, wherein the carbocycle is an aliphatic carbocycle.

4. The fluorine-containing ether compound according to claim 1, wherein a weight-average molecular weight Mw is no lower than 1,500.

5. A fluorine-containing ether composition comprising:
one or more kinds of the fluorine-containing ether compound according to claim 1; and
another fluorine-containing ether compound.

6. A coating liquid comprising:
the fluorine-containing ether compound according to claim 1 and
a liquid medium.

7. A coating liquid comprising:
the fluorine-containing ether composition according to claim 5 and
a liquid medium.

8. A high oxygen solubility liquid comprising:
the fluorine-containing ether compound according to claim 1; and
a liquid medium.

9. An article comprising:
a substrate having a surface with a surface layer formed by the fluorine-containing ether compound according to claim 1.

10. An article comprising:
a substrate having a surface with a surface layer formed by the fluorine-containing ether composition according to claim 5.
